(19) United States
(12) Patent Application Publication
(10) Pub. No.: US 2020/0288683 A1
(43) Pub. Date: Sep. 17, 2020

(54) LOSS OF FUNCTION RODENT MODEL OF SOLUTE CARRIER 39 MEMBER 5

(71) Applicant: Regeneron Pharmaceuticals, Inc., Tarrytown, NY (US)

(72) Inventors: Harikiran NISTALA, Tarrytown, NY (US); Aristides N. ECONOMIDES, Tarrytown, NY (US)

(21) Appl. No.: 16/816,826

(22) Filed: Mar. 12, 2020

Related U.S. Application Data
(60) Provisional application No. 62/818,872, filed on Mar. 15, 2019.
(60) Provisional application No. 62/976,437, filed on Feb. 14, 2020.

(51) Int. Cl. A01K 67/027

(52) U.S. Cl. CPC A01K 67/0276; A01K 2207/05; A01K 2227/105; A01K 2217/15; A01K 2217/077

(57) ABSTRACT

This disclosure relates to a rodent model. More specifically, this disclosure relates to a loss of function of solute carrier 39 member 5 (SLC39A5) rodent model. In particular, disclosed herein are genetically modified rodent animals that carry a loss of function mutation in an endogenous Slc39a5 gene and use of such rodent animals in elucidating the role of SLC39A5 in zinc homeostasis, glycemic regulation and lipid metabolism.

### CROSS REFERENCE TO RELATED APPLICATIONS

This application claims the benefit of priority from U.S. Provisional Application No. 62/818,872, filed Mar. 15, 2019 and U.S. Provisional Application No. 62/976,437, filed Feb. 14, 2020, the entire contents of which are incorporated herein by reference.

### FIELD OF THE DISCLOSURE

This disclosure relates to a rodent model. More specifically, this disclosure relates to a loss of function of solute carrier 39 member 5 (SLC39A5) rodent model. In particular, disclosed herein are genetically modified rodent animals that carry a loss of function mutation in an endogenous Slc39a5 gene and use of such rodent animals in elucidating the role of SLC39A5 in zinc homeostasis, glycemic regulation and lipid metabolism.

### INCORPORATION BY REFERENCE OF THE SEQUENCE LISTING

The sequence listing in the ASCII text file, named as 36843_10535US01_SequenceListing of 23 KB, created on Mar. 11, 2020, and submitted to the United States Patent and Trademark Office via EFS-Web, is incorporated herein by reference.

### BACKGROUND

Various references, including patents, patent applications, accession numbers, technical articles, and scholarly articles are cited throughout the specification. Each reference is incorporated by reference herein, in its entirety and for all purposes.

Zinc homeostasis is tightly controlled reflecting the essential roles zinc plays in the functions of a vast array of proteins. Impaired zinc metabolism is prominent in chronic disorders including cardiovascular diseases and diabetes. Randomized placebo-controlled zinc supplementation trials in humans demonstrated improved glycemic traits in patients with type II diabetes. Zinc supplementation has also been shown to reverse fatty liver disease in rodents.

Uptake and efflux of zinc involve two families of zinc transporters: members of the Slc39a or Zip family are believed to transport zinc into the cytoplasm of cells (either from extracellular milieu or from the vesicular compartments), and members of the Slc30a or ZnT family are believed to efflux zinc out of the cytosol (either into the extracellular milieu or into the vesicular compartment).

### SUMMARY OF THE DISCLOSURE

Disclosed herein are rodents (e.g., mice and rats) whose genome comprises a loss of function mutation in an endogenous Slc39a5 gene, and isolated rodent cells (e.g., ES cells) or tissues comprising a loss of function mutation in an endogenous Slc39a5 gene. Also disclosed herein are compositions (e.g., targeting vectors) and methods for the production of the rodents whose genome comprises a loss of function mutation in an endogenous Slc39a5 gene. Further disclosed herein are methods of using the rodents as an animal model of zinc homeostasis, glycemic regulation and lipid metabolism.

In one aspect, disclosed herein is a rodent whose genome comprises a loss of function mutation in an endogenous Slc39a5 gene at an endogenous rodent Slc39a5 locus. A loss of function mutation in an endogenous Slc39a5 gene at an endogenous rodent Slc39a5 locus results in the lack of a functional Slc39a5 polypeptide being expressed from the Slc39a5 locus, and elevation in circulating zinc levels in the rodent.

In some embodiments, a loss of function mutation comprises a point mutation in an exon of an endogenous rodent Slc39a5 gene. In some embodiments, a loss of function mutation comprises a deletion, in whole or in part, of the coding sequence of an endogenous rodent Slc39a5 gene. In some embodiments, the deletion comprises exon 1 in whole or in part, and/or exon 2 in whole or in part, of an endogenous rodent Slc39a5 gene. In some embodiments, the deletion comprises a coding portion of exon 1 and a portion of exon 2 of an endogenous rodent Slc39a5 gene. In some embodiments, the deletion comprises a nucleic acid sequence from the nucleotide after the ATG start codon in exon 1 through the fifth nucleotide before the 3′ end of exon 2 of an endogenous rodent Slc39a5 gene.

In some embodiments, the rodent Slc39a5 locus which comprises a loss of function mutation in an endogenous Slc39a5 gene further comprises a reporter gene.

In some embodiments, the reporter gene is operably linked to the endogenous Slc39a5 promoter at the Slc39a5 locus. In specific embodiments, the Slc39a5 locus comprises a deletion beginning from the nucleotide after the ATG start codon in exon 1 through the fifth nucleotide before the 3′ end of exon 2 of the endogenous rodent Slc39a5 gene, and comprises a reporter gene coding sequence that is fused in-frame to the start (ATG) codon of the Slc39a5 locus.

In some embodiments, the reporter gene is lacZ. In some embodiments, the reporter gene is selected from the group consisting of luciferase, green fluorescent protein (GFP), enhanced GFP (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

In some embodiments, a rodent is homozygous for a loss of function mutation in an endogenous Slc39a5 gene. In some embodiments, a rodent is heterozygous for a loss of function mutation in an endogenous Slc39a5 gene.

In some embodiment, a rodent is a male rodent. In some embodiments, a rodent is a female rodent.

In some embodiments, a rodent is a mouse. In some embodiments, a rodent is a rat.

In some embodiments, a rodent is a female rodent, e.g., a female mouse, that is homozygous for a loss of function mutation in an endogenous Slc39a5 gene. Such a female rodent exhibits elevation in circulating zinc levels as compared to wild type rodents without a loss of function mutation in an endogenous Slc39a5 gene. In some embodiments, such a female rodent exhibit reduced fasting blood sugar levels as compared to littermate controls. In some embodiments, such as female rodent exhibits elevated hepatic zinc levels and improvements in serum alanine aminotransferase (ALT) and aspartate aminotransferase (AST) levels (biomarkers of liver injury) on a high fat diet as compared to wild type rodents on a high fat diet.

In some embodiments, a rodent whose genome comprises a loss of function mutation in an endogenous Slc39a5 gene further comprises a loss of function mutation in an endogenous rodent leptin receptor gene (Lepr). The loss of function mutation in an endogenous rodent Lepr gene results in the lack of a functional leptin receptor being expressed. Leptin-receptor deficient rodents have been established as a rodent model of obesity induced type II diabetes. Introduction of a loss of function mutation in an endogenous Slc39a5 gene into a leptin-receptor deficient rodent leads to the rescue of chronic hyperglycemia resulting from the leptin-receptor deficiency.

In some embodiments, a loss of function mutation in an endogenous rodent Lepr gene comprises a point mutation in an exon of an endogenous rodent leptin receptor gene. In some embodiments, a loss of function mutation comprises a deletion, in whole or in part, of the coding sequence of an endogenous rodent leptin receptor gene. In some embodiments, the deletion includes a nucleotide sequence of an endogenous rodent Lepr gene encoding the extracellular domain in whole or in part. In some embodiments, the deletion comprises exon 1, exon 2, exon 3, exon 4, exon 5, and/or exon 6, each in whole or in part, of an endogenous rodent Lepr gene. In some embodiments, the deletion comprises a coding portion of exon 1, and exons 2-6, of an endogenous rodent Lepr gene.

Also provided herein is a progeny of any of the rodents disclosed herein.

In a further aspect, disclosed herein is an isolated rodent cell or tissue whose genome comprises a loss of function mutation in an endogenous rodent Slc39a5 gene at an endogenous rodent Slc39a5 locus.

In some embodiments, the isolated rodent cell is a rodent embryonic stem cell, or a rodent egg.

In another aspect, disclosed herein is a rodent embryo whose genome comprises a loss of function mutation in an endogenous rodent Slc39a5 gene at an endogenous rodent Slc39a5 locus.

In still a further aspect, disclosed herein is a method of making a rodent whose genome comprises a loss of function mutation in an endogenous rodent Slc39a5 gene at an endogenous rodent Slc39a5 locus. The method comprises modifying a rodent genome such that the modified rodent genome comprises a loss of function mutation in an endogenous rodent Slc39a5 gene at an endogenous rodent Slc39a5 locus, and obtaining a rodent comprising the modified genome.

In some embodiments, a rodent genome is modified by introducing a nucleic acid sequence into the genome of a rodent embryonic stem cell, which nucleic acid sequence comprises polynucleotide sequences that are homologous to nucleic acid sequences at the endogenous rodent Slc39a5 locus, such that the modified genome comprises a loss of function mutation in an endogenous rodent Slc39a5 gene at the Slc39a5 locus, thereby obtaining a genetically modified rodent embryonic stem cell, and making a rodent using the genetically modified rodent embryonic stem cell.

In some embodiments, the loss of function mutation is a rodent made by the method comprises a point mutation in an exon of an endogenous rodent Slc39a5 gene. In some embodiments, a loss of function mutation comprises a deletion, in whole or in part, of the coding sequence of an endogenous rodent Slc39a5 gene. In some embodiments, the deletion comprises exon 1 in whole or in part, and/or exon 2 in whole or in part, of an endogenous rodent Slc39a5 gene. In some embodiments, the deletion comprises a coding portion of exon 1 and a portion of exon 2 of an endogenous rodent Slc39a5 gene. In some embodiments, the deletion comprises a nucleic acid sequence from the nucleotide after the ATG start codon in exon 1 through the fifth nucleotide before the 3′ end of exon 2 of an endogenous rodent Slc39a5 gene.

In some embodiments, a rodent made by the present method further comprises a reporter gene. For example, a reporter gene can be included in the nucleic acid sequence being introduced into the genome of a rodent embryonic stem cell. In some embodiments, the reporter gene is operably linked to the endogenous Slc39a5 promoter at the Slc39a5 locus in the modified genome. In specific embodiments, the Slc39a5 locus of a modified genome comprises a deletion beginning from the nucleotide after the ATG start codon in exon 1 through the fifth nucleotide before the 3′ end of exon 2 of the endogenous rodent Slc39a5 gene, and comprises a reporter gene coding sequence that is fused in-frame to the start (ATG) codon of the Slc39a5 locus.

In some embodiments, the reporter gene is lacZ. In some embodiments, the reporter gene is selected the group consisting of luciferase, green fluorescent protein (GFP), enhanced GFP (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

In some embodiments, a rodent made by the present method further comprises a selection marker. For example, a selection marker gene can be included in the nucleic acid sequence being introduced into the genome of a rodent embryonic stem cell. In some embodiments, the nucleic acid sequence may further comprise site-specific recombinase recognition sites flanking the selection marker gene, which site-specific recombinase recognition sites are oriented to direct an excision of the selection marker by a recombinase.

In some embodiments, a rodent made by the present method is heterozygous for a loss of function mutation in an endogenous Slc39a5 gene. Rodents heterozygous for a loss of function mutation in an endogenous Slc39a5 gene can be bred with each other to obtain rodents homozygous for the loss of function mutation in an endogenous Slc39a5 gene.

In some embodiment, a rodent made by the present method is a male rodent. In some embodiments, a rodent made by the present method is a female rodent.

In some embodiments, a rodent made by the present method is a mouse. In some embodiments, a rodent made by the present method is a rat.

In a further aspect, disclosed herein is a targeting nucleic acid construct, comprising a nucleic acid sequence to be integrated into a rodent Slc39a5 gene at an endogenous rodent Slc39a5 locus, flanked by a 5′ nucleotide sequence and a 3′ nucleotide sequence that are homologous to nucleotide sequences at the rodent Slc39a5 locus, wherein integration of the nucleic acid sequence into the rodent Slc39a5 gene results in a loss of function mutation in the endogenous rodent Slc39a5 gene as described herein. The targeting nucleic acid construct can be designed for integrating the nucleic acid sequence into a mouse or rat Slc39a5 gene at an endogenous mouse or rat Slc39a5 locus. In some embodiments, the nucleic acid sequence to be integrated into a rodent Slc39a5 gene at an endogenous rodent Slc39a5 locus includes a reporter gene. In some embodiments, the nucleic acid sequence to be integrated into a rodent Slc39a5 gene at an endogenous rodent Slc39a5 locus includes a selectable marker gene.

In a further aspect, disclosed herein is a method of breeding, comprising breeding a first rodent whose genome comprises a loss of function mutation in an endogenous rodent Slc39a5 gene, with a second rodent, resulting in a progeny rodent whose genome comprises the loss of function mutation in an endogenous rodent Slc39a5 gene.

In some embodiments, the second rodent comprises a loss of function mutation in an endogenous rodent leptin receptor gene (Lepr). In some embodiments, a loss of function mutation in an endogenous rodent Lepr gene comprises a point mutation in an exon of an endogenous rodent Lepr gene. In some embodiments, a loss of function mutation comprises a deletion, in whole or in part, of the coding sequence of an endogenous rodent Lepr gene. In some embodiments, the deletion comprises a nucleotide sequence of an endogenous rodent Lepr gene encoding the extracellular domain in whole or in part. In some embodiments, the deletion comprises exon 1, exon 2, exon 3, exon 4, exon 5, and/or exon 6, each in whole or in part, of an endogenous rodent Lepr gene. In some embodiments, a loss of function mutation comprises a deletion of the coding portion of exon 1 through exon 6 of an endogenous rodent Lepr gene, and an insertion of a reporter gene.

In a further aspect, disclosed herein is use of a rodent whose genome comprises a loss of function mutation in an endogenous Slc39a5 gene as an animal model, which permits elucidation of the mechanisms of Slc39a5 action in the context of glycemic regulation and provides opportunities to test and develop therapeutics to target Slc39a5 in the treatment of metabolic and cardiovascular disorders.

In some embodiments, a rodent whose genome comprises a loss of function mutation in an endogenous Slc39a5 gene is used in a method of testing, screening, or identifying an agent that inhibits the activity of a Slc39a5 protein. In accordance with such method, a rodent whose genome comprises a loss of function mutation in an endogenous Slc39a5 gene is used along with a wild type rodent without the mutation, and a candidate agent is administered to the wild type rodent. Both the wild type rodent and the rodent with the loss of function mutation are examined to measure the serum zinc levels and one or more metabolic and cardiovascular traits, including one or more liver, lipid or glycemic traits. The measurements from the wild type rodent after the administration of the agent, from the wild type rodent before the administration (or from another wild type rodent not administered the agent), and from the rodent with the loss of function mutation, are compared with one another to determine whether the agent inhibits the activity of a Slc39a5 protein.

In some embodiments, at least one of the traits measured is the serum level of alanine aminotransferase and/or aspartate aminotransferase after the rodents are fed with a high fat diet, in some embodiments, the high fat diet is a high fat high fructose diet (“HFFD”). In some embodiments, at least one of the traits measured is hepatic steatosis of liver sections after the rodents are fed with high fat diet, e.g., a HFFD. In some embodiments, at least one of the traits measured is the fasting glucose level in the serum. In some embodiments, at least one of traits measured is body weight. In some embodiments, at least one of traits measured is the level of low density lipoprotein (LDL) or the level of high density lipoprotein (HDL). In some embodiments, the rodents are also examined to measure the zinc level in the serum.

Agents that have been identified to inhibit the activity of a Slc39a5 protein may be used in the treatment of metabolic and cardiovascular disorders including, for example, increased serum glucose level, hyperglycemia, Type 2 diabetes, obesity, increased low density lipoprotein (LDL), decreased high density lipoprotein (HDL), alcoholic fatty-liver disease, non-alcoholic fatty liver disease, non-alcoholic steatohepatitis, and hepatic encephalopathy.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIGS. 1A-B depict the domain and topological structures of the human SLC39A5 protein. Transmembrane Domains (TM) are labeled as is the signal peptide spanning the first 20 amino acids. Topological extracellular and cytoplasmic domains are rendered in red and yellow, respectively.

FIG. 2 sets forth alignment of human, mouse and rat SLC39A5 protein sequences (SEQ ID NOS: 2, 4 and 6, respectively). Asterisks (*) denote amino acid residues shared by all three sequences.

FIG. 3 depicts an exemplary targeting strategy for generating Slc39a5 gene ablating deletion with a β-galactosidase (lacZ) reporter cassette into a wild-type Slc39a5 allele in a mouse. Asterisks (*) represent loss of allele (LOA) or gain of allele (GOA) used for genotyping.

FIGS. 4A-C depict body weight, fasting blood glucose and serum zinc levels of Slc39a5 loss-of-function mice at 12 weeks of age. (A) Both male and female homozygous loss-of-function mice had slightly reduced body weight as compared to heterozygous loss-of-function and wild-type mice at 12 weeks. (B) Female homozygous loss-of-function mice had reduced fasting blood glucose levels as compared to heterozygous loss-of-function and wild-type mice at 12 weeks of age. No differences were observed in male mice at this age. (C) Both male and female heterozygous and homozygous loss-of-function mice had significantly elevated serum zinc levels as compared to wild-type mice at 12 weeks of age.

FIGS. 5A-B depict serum zinc, liver zinc, alanine aminotransferase and aspartate aminotransferase levels of Slc39a5 loss-of-function mice at 40 weeks on a high-fat diet regimen. (A) Female homozygous loss-of-function mice had elevated serum zinc and liver zinc levels as compared to wild-type mice on a normal chow (NC) or a high-fat diet (HFD). Furthermore, female homozygous loss-of-function mice had reduced alanine aminotransferase and asparate aminotransferase levels as compared to wild-type mice on a high-fat diet (HFD). (B) Male homozygous loss-of-function mice had elevated serum zinc levels as compared to wild-type mice on a normal chow (NC) or a high-fat diet (HFD) at 40 weeks. No statistically significant differences were observed in liver zinc, alanine aminotransferase and aspartate aminotransferase levels in male homozygous loss-of-function mice as compared to wild-type mice on a normal chow (NC) or a high-fat diet (HFD).

FIGS. 6A-B show histological analyses of liver from Slc39a5 loss-of-function mice at 40 weeks on a high-fat diet regimen. (A) Histochemical comparison of hematoxylin and eosin stained liver sections demonstrated that female homozygous loss-of-function mice on a high fat diet had reduced hepatic steatosis as compared to wild-type mice on a high-fat diet (HFD). (B) Histochemical comparison of hematoxylin and eosin stained liver sections demonstrated no overt differences between male homozygous loss-of-function mice on a high fat diet as compared to wild-type mice on a high-fat diet (HFD).

FIGS. 7A-B depict serum chemistry and oral glucose tolerance test results of female mice homozygous for Slc39a5 and leptin-receptor (Lepr) loss-of-function mutations on normal chow at 20 weeks of age. (A) Mice homozygous for Slc39a5 and Lepr loss-of-function mutations had reduced alanine aminotransferase, aspartate aminotransferase, low-density lipoprotein cholesterol and fasting blood glucose levels as compared to Lepr loss of function mice at 20 weeks. (B) Furthermore, female mice homozygous for Slc39a5 and Lepr loss-of-function mutations displayed improved glucose tolerance as compared to Lepr loss of function mice at 20 weeks.

FIG. 8. Loss of function of Slc39a5 improves fasting blood glucose upon high fat high fructose dietary challenge. High fat high fructose dietary challenge results in significant increase in body weight across all genotypes in both sexes. Loss of function of Slc39a5 improves liver function as assessed by serum ALT and AST in both sexes at 16 weeks. Importantly, loss of function of Slc39a5 significantly improves hyperglycemia assessed by fasting blood glucose levels at endpoint (29 weeks). “NC”: normal chow (NC); “HFFD”: high fat high fructose diet. Body weight—27 weeks; Blood Glucose Measures: Fed—25 weeks and Fast (16 hr fast)-29 weeks; Zinc 34 ppm; *p<0.05, **p<0.01; Error bars: SEM.

FIG. 9. Loss of function of Slc39a5 improves insulin sensitivity in mice challenged with high fat high fructose diet. Mice homozygous for Slc39a5 loss of function (regardless of sex) show marked improvement in insulin sensitivity compared to wild type counterparts assessed by oral glucose tolerance tests (“GTT”). High fat high fructose (HFFD) or Normal Chow (NC) for 18 weeks; Zinc 34 ppm; oGTT after 16 hr fast, 2 mg/g body weight; *p<0.05, **p<0.01.

FIG. 10. Loss of function of Slc39a5 improves hepatic steatosis upon high fat high fructose dietary (“HFFD”) challenge. Slc39a5 loss of function female mice are more protected than their male counterparts. NAFLD composite scores (assessed by two independent pathologists) representing an aggregate score of macrovesicular steatosis, microvesicular steatosis, hepatocellular hypertrophy, inflammation and fibrosis show a significant improvement in female Slc39a5 knockout mice as compared to wild type counterparts; whereas in male mice, loss of Slc39a5 improves hepatic steatosis on normal chow and accords no protection when challenged by high fat high fructose diet demonstrated by histopathology and NAFLD scores. Liver histology—29 weeks; NAFLD Composite Score: Macrovesicular Steatosis, Microvesicular Steatosis, Hepatocellular Hypertrophy, Inflammation, Fibrosis; *p<0.05, **p<0.01; Error bars: SEM.

FIGS. 11A-1B. Loss of function of Slc39a5 results in increased hepatic zinc levels and a consequent elevation in hepatic metallothionein (“Mt1” and “Mt2”) expression. Furthermore, loss of function of Slc39a5 does not significantly influence hepatic iron, copper, cobalt, calcium and magnesium levels. 11A: female mice; 11B: male mice. Hepatic ion quantification and Taqman analysis—29 weeks; *p<0.05, **p<0.01; Error bars: SEM.

FIGS. 12A-12B. Zinc acutely activates LKB1/AMPK and AKT signaling pathways in dose dependent manner in human hepatoma HepG2 cells (12A) and human primary hepatocytes (12B). 1° Hu Hepatocytes* (5-donor pool): HM CPP5, Thermofisher.

FIG. 13A. Loss of function of Slc39a5 improves hepatic steatosis in female mice challenged with high fat high fructose diet. Furthermore, loss of function of Slc39a5 results in increased hepatic zinc levels with concomitant activation of hepatic AMPK and AKT signaling in female mice challenged with high fat high fructose diet. In support of these observations, hepatic triglyceride levels were reduced with an increase in hepatic beta-hydroxybutyrate levels, suggesting increased β-oxidation. Moreover, loss of function of Slc39a5 results in a downregulation of Fasn and G6pc genes involved in denovo lipogenesis and hepatic gluconeogenesis, respectively. Liver lysates—29 weeks; *p<0.05, **p<0.01; Error bars: SEM.

FIG. 13B. Loss of function of Slc39a5 improves hepatic steatosis in male mice fed with normal chow. Furthermore, loss of Slc39a5 in male mice challenged with high fat high fructose diet results in insignificant elevation in hepatic zinc levels with a modest activation of hepatic AMPK signaling. Hepatic triglyceride levels are slightly reduced with an increase in hepatic beta-hydroxybutyrate levels. Moreover, loss of function of Slc39a5 results in a modest repression of Fasn and significant downregulation of G6pc, genes involved in denovo lipogenesis and hepatic gluconeogenesis, respectively. Liver lysates—29 weeks; *p<0.05, **p<0.01; Error bars: SEM.

FIG. 14. Loss of function of Slc39a5 improves liver function and fasting blood glucose in leptin-receptor deficient mice. Congenital leptin-receptor deficiency results in significant increase in body weight in Lepr^{-/-} and Slc39a5^{-/-}; Lepr^{-/-} mice in both sexes. Loss of function of Slc39a5 improves liver function in leptin-receptor deficient mice (both sexes) as assessed by serum ALT and AST at 22 weeks. Importantly, loss of function of Slc39a5 significantly improves hyperglycemia in leptin-receptor deficient mice (both sexes) demonstrated by reduced fasting blood glucose levels at 34 weeks. Normal Chow (Zinc 87 ppm); Fasting blood glucose: 32 weeks (Fed) and 34 weeks (16 hr/Fast); *p<0.05, **p<0.01; Error bars: SEM.

FIG. 15. Loss of function of Slc39a5 in leptin-receptor deficient mice (both sexes) results in improved insulin sensitivity as compared to leptin-receptor deficient (Lepr^{-/-}) counterparts assessed by oral glucose tolerance tests (“GTT”). Normal chow; Zinc 87 ppm; oGTT (20 wk) after 16 hr fast—2 mg/g body weight; *p<0.05, **p<0.01; Error bars: SEM.

FIG. 16. Loss of function of Slc39a5 improves hepatic steatosis in leptin-receptor deficient mice (both sexes).

FIG. 17. Loss of function of Slc39a5 results in increased hepatic zinc levels and a consequent elevation in hepatic metallothionein (“Mt1” and “Mt2”) expression in leptin-receptor deficient mice (both sexes). Furthermore, loss function of Slc39a5 does not significantly influence hepatic iron levels in these mice. Hepatic ion quantification and Taqman analysis—29 weeks; *p<0.05, **p<0.01; Error bars: SEM.

FIGS. 18A-18B. Loss of function of Slc39a5 improves hepatic steatosis in leptin receptor deficient mice in both sexes (18A, female; 18B male). Furthermore, loss of function of Slc39a5 results in increased hepatic zinc levels in leptin receptor deficient mice (both sexes) with concomitant activation of hepatic AMPK signaling. In support of these observations, hepatic triglyceride levels are reduced with an increase in hepatic beta-hydroxybutyrate levels suggesting increased β-oxidation. Moreover, loss of Slc39a5 results in a downregulation of Fasn indicative of reduced denovo lipogenesis. Liver lysates—29 weeks; *p<0.05, **p<0.01; Error bars: SEM.

### DETAILED DESCRIPTION

Disclosed herein is a rodent model for loss of function of SLC39A5. In particular, disclosed herein are genetically modified rodent animals that carry a loss of function mutation in an endogenous rodent Slc39a5 gene. In line with observations in humans that heterozygous loss of function carriers of European ancestry were associated with elevated serum zinc levels and protection against type II diabetes, it has been demonstrated herein that both homozygous and heterozygous inactivation of an endogenous Slc39a5 gene result in elevation in circulating zinc levels in rodent animals. Furthermore, it has been shown herein that female mice homozygous for Slc39a5 loss of function exhibit (i) reduced fasting blood sugar levels as compared to littermate controls, and (ii) elevated hepatic zinc levels and improvements in serum ALT and AST levels (biomarkers of liver injury) on a high fat diet as compared to wild type rodents on a high fat diet. Moreover, it has been shown herein that a loss of function of Slc39a5 in leptin-receptor deficient mice (a murine model of obesity induced type II diabetes) results in the rescue of chronic hyperglycemia. Accordingly, the engineered rodent model provided herein recapitulates the SLC39A5 loss of function phenotype in humans, thereby providing a valuable model to elucidate and develop SLC39A5 inhibitory therapeutics to treat glycemic dysregulation, metabolic and cardiovascular disorders.

Various aspects of the present disclosure are described in detail below.

SLC39A5 encodes solute carrier 39 member 5, a zinc transporter crucial in controlling cellular zinc levels. SLC39A5 is primarily expressed in the small intestine, kidney, liver and pancreas and is thought to regulate zinc homeostasis.

Exemplary mRNA and protein sequences from human, mouse and rat are available in GenBank under the following accession numbers, and are also set forth as SEQ ID NOS: 1-6 in the Sequence Listing.

TABLE 1

| SEQ ID NO | Description | Features |
|---|---|---|
| 1 | Homo sapiens SLC39A5 mRNA | Length: 1980 bp |
| | (Genomic context: NC_000012.12) | |
| 2 | Homo sapiens SLC39A5 protein | Length: 540 aa |
| 3 | Mus musculus Slc39a5 mRNA | Length: 1944 bp |
| | (Genomic context: NC_000076.6) | |
| 4 | Mus musculus Slc39a5 protein | Length: 535 aa |
| 5 | Rattus norvegicus Slc39a5 mRNA | Length: 2828 bp |
| | (Genomic context: NC_005106.4) | |
| 6 | Rattus norvegicus Slc39a5 protein | Length: 533 aa |

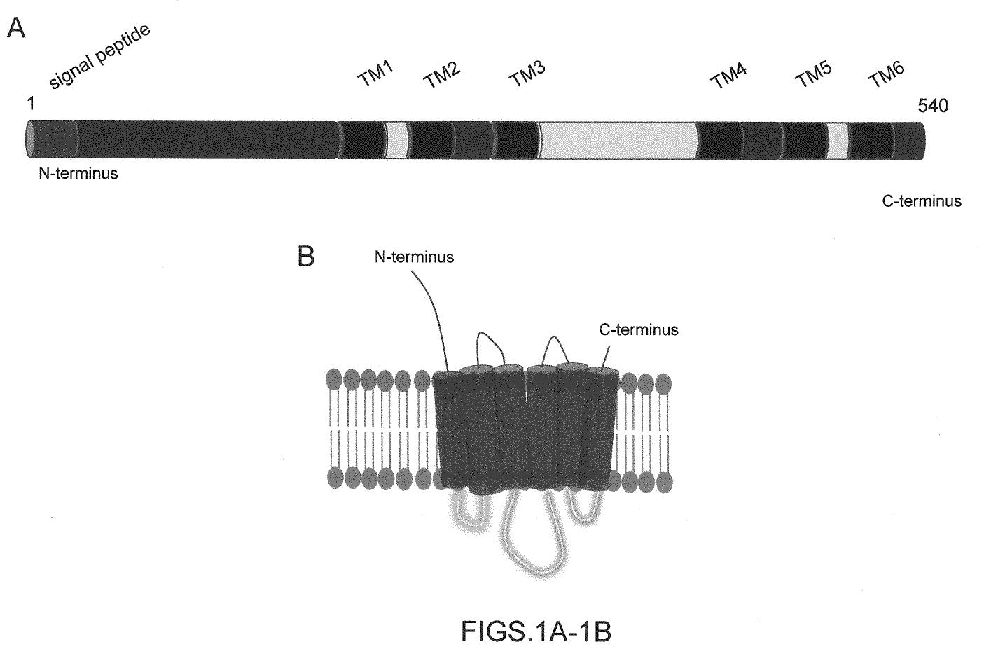

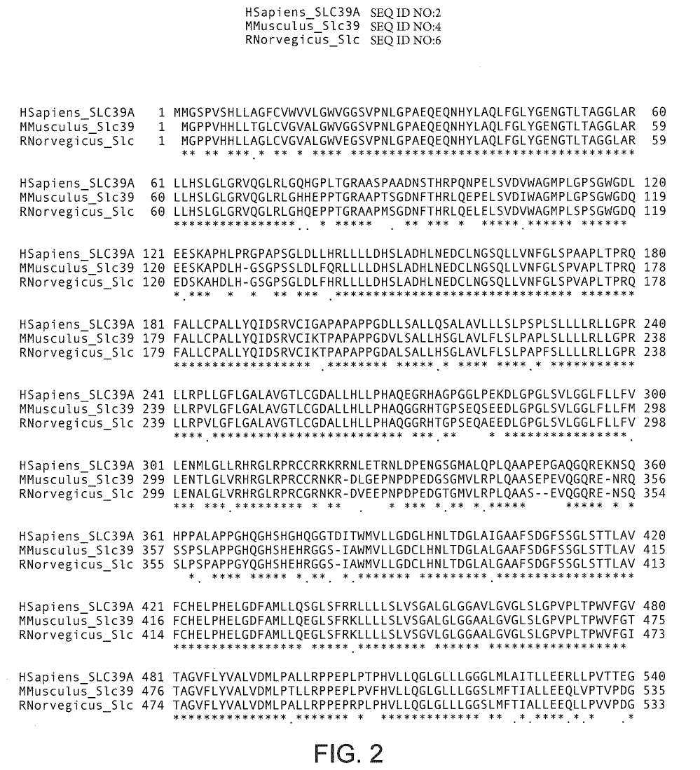

The protein structure is well conserved across species. As shown in FIG. 1A-1B, the SLC39A5 protein contains a signal peptide, an N-terminal extracellular segment, and six transmembrane segments which are connected to each other by three cytoplasmic domains (loops) and two extracellular domains (loops).

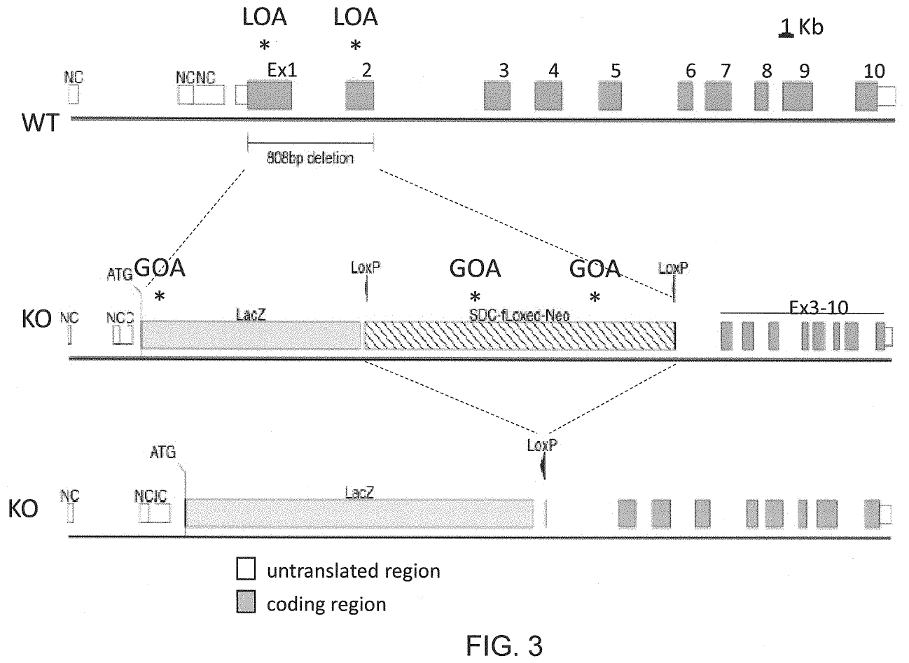

The genomic structure is also conserved across species. To illustrate, FIG. 3 depicts the genomic structure of mouse Slc39a5 gene as consisting of ten coding exons, with the first coding exon being designated as exon 1.

Disclosed herein are rodents (e.g., mice and rats) whose genome comprises a loss of function mutation in an endogenous Slc39a5 gene.

The term “mutation” includes an addition, deletion, or substitution of one or more nucleotides in a gene. As used herein, the terms “mutation”, “alteration”, and “variation” are used interchangeably. A mutant gene (or a mutant allele of a gene) is understood herein to include a mutation, alteration or variation relative to a wild type gene or a reference gene. In some embodiments, a mutation is a substitution of a single nucleotide. In other embodiments, a mutation is a deletion of one or more nucleotides, e.g., one or more nucleotides in the coding sequence of a gene. In some embodiments, a mutation in a gene includes a deletion of a contiguous nucleic acid sequence, e.g., one or more exons or all exons, the coding sequence in full or in part, of a gene. In some embodiments, a mutation in a gene results in an addition, deletion, or substitution of one or more amino acids in the encoded protein.

In some embodiments, a mutation is a loss of function mutation. As used herein, the term “loss of function” includes a complete loss of function and a partial loss of function. In some embodiments, an alteration in a gene results in expression of a polypeptide with at least diminished functionality and, in some cases, with a substantially diminished functionality or complete lack of functionality relative to a polypeptide encoded by a reference gene not having the alteration. Thus, a genetic alteration may cause a complete loss of function or a partial loss of function.

In some embodiments, a loss of function mutation in a Slc39a5 gene includes a deletion of the first coding exon (i.e., exon 1) in whole or in part, e.g., the coding portion of exon 1 beginning from the nucleotide after the ATG codon. In some embodiments, a loss of function mutation in a Slc39a5 gene includes a deletion of the second or subsequent coding exon in whole or in part. In some embodiments, a loss of function mutation in a Slc39a5 gene includes a deletion of the coding sequence of exon 1 beginning from the nucleotide after the ATG codon and a deletion of the second coding exon in whole or in part. In some embodiments, a loss of function mutation in a Slc39a5 gene includes a deletion of the sequence(s) encoding one or more or all of the transmembrane domains.

In some embodiments, a rodent whose genome comprises a loss of function mutation in an endogenous Slc39a5 gene further comprises an insertion of a reporter gene, and wherein the reporter gene is operably linked to the endogenous rodent Slc39a5 promoter at the locus.

In some embodiments, a genomic fragment beginning from the nucleotide after the start codon in the first coding exon through the whole or part of a subsequent coding exon (e.g., the second, third, fourth, fifth, sixth, seventh, eighth, ninth or tenth coding exon) has been deleted, and the reporter gene is inserted immediately downstream of the start codon of the endogenous rodent Slc39a5 gene. In such linkage, expression of the reporter gene is expected to resemble the expression pattern of an unmodified endogenous rodent Slc39a5 gene.

Multiple reporter genes are known in the art and are suitable for use herein. In some embodiments, the reporter gene is a LacZ gene. In some embodiments, the reporter gene is a gene encoding a protein selected the group consisting of luciferase, green fluorescent protein (GFP), enhanced GFP (eGFP), cyan fluorescent protein (CFP), yellow fluorescent protein (YFP), enhanced yellow fluorescent protein (eYFP), blue fluorescent protein (BFP), enhanced blue fluorescent protein (eBFP), DsRed, and MmGFP.

For any of the embodiments described herein, the rodents can include, for example, mice, rats, and hamsters.

In some embodiments, the rodent is a mouse. In some embodiments, the rodent is a mouse of a C57BL strain, for example, a C57BL strain selected from C57BL/A, C57BL/An, C57BL/GrFa, C57BL/KaLwN, C57BL/6, C57BL/6J, C57BL/6ByJ, C57BL/6NJ, C57BL/10, C57BL/10ScSn, C57BL/10Cr, and C57BL/Ola. In other embodiments, the rodent is a mouse of a 129 strain, for example, a 129 strain selected from the group consisting of 129P1, 129P2, 129P3, 129X1, 129S1 (e.g., 129S1/SV, 129S1/SvIm), 129S2, 129S4, 129S5, 129S9/SvEvH, 129/SvJae, 129S6 (129/SvEvTac), 129S7, 129S8, 129T1, 129T2 (see, e.g., Festing et al. (1999), Mammalian Genome 10:836; Auerbach et al. (2000), Biotechniques 29(5): 1024-1028, 1030, 1032). In some embodiments, the rodent is a mouse that is a mix of an aforementioned 129 strain and an aforementioned C57BL/6 strain. In certain embodiments, the mouse is a mix (i.e., hybrid) of aforementioned 129 strains, or a mix of aforementioned C57BL strains, or a mix of a C57BL strain and a 129 strain. In certain embodiments, the mouse is a mix of a C57BL/6 strain with a 129 strain. In specific embodiments, the mouse is a VGF1 strain, also known as F1H4, which is a hybrid of C57BL/6 and 129. In other embodiments, the mouse is a BALB strain, e.g., BALB/c strain. In some embodiments, the mouse is a mix of a BALB strain and another aforementioned strain.

In some embodiments, the rodent is a rat. In certain embodiments, the rat is selected from a Wistar rat, an LEA strain, a Sprague Dawley strain, a Fischer strain, F344, F6, and Dark Agouti. In other embodiments, the rat is a mix of two or more strains selected from the group consisting of Wistar, LEA, Sprague Dawley, Fischer, F344, F6, and Dark Agouti.

Also disclosed herein are isolated rodent cells or tissues comprising a loss of function mutation in an endogenous Slc39a5 gene, described herein. In some embodiments, an isolate rodent cell is an embryonic stem (ES) cell. Rodent embryos and eggs comprising a loss of function mutation in an endogenous Slc39a5 gene are also provided.

Disclosed herein are methods for the production of the rodents having a loss of function mutation in an endogenous Slc39a5 gene.

The method comprises modifying a rodent genome such that the modified rodent genome comprises a loss of function mutation in an endogenous rodent Slc39a5 gene at an endogenous rodent Slc39a5 locus, and obtaining a rodent comprising the modified genome.

In some embodiments, a rodent genome is modified by, e.g., employing a zinc finger nuclease (ZFN), a transcription activator-like effector nuclease (TALEN), or a Cas protein (i.e., a CRISPR/Cas system), such that the modified genome includes a loss of function mutation in an endogenous rodent Slc39a5 gene at an endogenous rodent Slc39a5 locus.

In some embodiments, a rodent genome is modified by introducing a nucleic acid sequence into the genome of a rodent embryonic stem (ES) cell, wherein the nucleic acid sequence comprises polynucleotide sequences that are homologous to nucleic acid sequences at the endogenous rodent Slc39a5 locus so as to be capable of mediating homologous recombination of the nucleic acid sequence into the genome of the ES cell, such that the modified genome comprises a loss of function mutation in an endogenous rodent Slc39a5 gene at the Slc39a5 locus, thereby obtaining a genetically modified rodent embryonic stem cell, and making a rodent using the genetically modified rodent embryonic stem cell.

In some embodiments, the nucleic acid sequence to be introduced (i.e., the insert nucleic acid) into the genome of a rodent ES cell is provided in a targeting nucleic acid construct (i.e., a targeting vector), preferably a DNA vector. In some embodiments, the insert nucleic acid also contains a selectable marker gene (e.g., a self deleting cassette containing a selectable marker gene, as described in U.S. Pat. Nos. 8,697,851, 8,518,392 and 8,354,389, all of which are incorporated herein by reference), which can be flanked by or comprises site-specific recombination sites (e.g., loxP, Frt, etc.). The selectable marker gene can be placed on the vector adjacent to the mutation to permit easy selection of transfectants. In some embodiments, the insert nucleic acid also contains a reporter gene.

In some embodiments, a targeting vector (e.g., a BAC vector) can be introduced into rodent embryonic stem (ES) cells by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference) describe mouse ES cells and the VELOCIMOUSE® method for making a genetically modified mouse; and US 2014/0235933 A1 and US 2014/0310828 A1 (all of which are incorporated herein by reference) describe rat ES cells and methods for making a genetically modified rat.

Homologous recombination in recipient cells can be facilitated by introducing a break in the chromosomal DNA at the integration site, which may be accomplished by targeting certain nucleases to the specific site of integration. DNA-binding proteins that recognize DNA sequences at the target locus are known in the art. In some embodiments, zinc finger nucleases (ZFNs), which recognize a particular 3-nucleotide sequence in a target sequence, are utilized. In some embodiments, Transcription activator-like (TAL) effector nucleases (TALENs) are employed for site-specific genome editing. In other embodiments, RNA-guided endonucleases (RGENs), which consist of components (Cas9 and tracrRNA) and a target-specific CRISPR RNA (crRNA), are utilized.

In some embodiments, a targeting vector carrying a nucleic acid of interest (e.g., a mutant rodent Slc39a5 gene sequence to be introduced), flanked by 5′ and 3′ homology arms, is introduced into a cell with one or more additional vectors or mRNA. In one embodiment, the one or more additional vectors or mRNA contain a nucleotide sequence encoding a site-specific nuclease, including but not limited to a zinc finger nuclease (ZFN), a ZFN dimer, a transcription activator-like effector nuclease (TALEN), a TAL effector domain fusion protein, and an RNA-guided DNA endonuclease.

ES cells having the mutant gene sequence integrated in the genome can be selected. After selection, positive ES clones can be modified, e.g., to remove a self-deleting cassette, if desired. ES cells having the mutation integrated in the genome can then be used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008/0078000 A1), or methods described in US 2014/0235933 A1 and US 2014/0310828 A1. The embryo comprising the donor ES cells is incubated until blastocyst stage and then implanted into a surrogate mother to produce an F0 rodent fully derived from the donor ES cells. Rodent pups bearing the mutant allele can be identified by genotyping of DNA isolated from tail snips using a modification of allele (MOA) assay (Valenzuela et al., supra) that detects the presence of the mutant sequence or a selectable marker gene.

Further provided herein are methods of breeding a genetically modified rodent as described herein with another rodent, as well as progenies obtained from such breeding.

In some embodiments, a method is provided which comprises breeding a first genetically modified rodent as described hereinabove (e.g., a rodent whose genome comprises a loss of function Slc39a5 mutation at an endogenous rodent Slc39a5 locus), with a second rodent, resulting in a progeny rodent whose genome comprises the loss of function Slc39a5 mutation. The progeny may possess other desirable phenotypes or genetic modifications inherited from the second rodent used in the breeding. In some embodiments, the progeny rodent is heterozygous for the loss of function Slc39a5 mutation. In some embodiments, the progeny rodent is homozygous for the loss of function Slc39a5 mutation.

In some embodiments, a progeny rodent is provided whose genome comprises a loss of function Slc39a5 mutation at an endogenous rodent Slc39a5 locus, wherein the progeny rodent is produced by a method comprising breeding a first genetically modified rodent as described hereinabove (e.g., a rodent whose genome comprises a loss of function Slc39a5 mutation at an endogenous rodent Slc39a5 locus), with a second rodent. In some embodiments, the progeny rodent is heterozygous for the loss of function Slc39a5 mutation. In some embodiments, the progeny rodent is homozygous for the loss of function Slc39a5 mutation.

In some embodiments, the second rodent comprises a loss of function mutation in an endogenous rodent leptin receptor gene (Lepr).

Leptin receptor belongs to the class I cytokine receptor family and exists in five different isoforms. Four of the five isoforms have an identical extracellular domain (responsible for ligand binding) and a transmembrane domain, but differ in the length and sequence of their intracellular C-terminal domains. The fifth isoform, known as the “soluble” or “secreted” isoform, contains no transmembrane domain, and is encoded by a fifth alternatively spliced transcript variant in rodents while being generated by proteolytic cleavage of the transmembrane isoforms in humans. The protein structure, the genomic organization of the Lepr gene, the mechanisms of action, and the association between leptin receptor deficiencies with obesity, are documented in the art (see, e.g., Dam et al., in Leptin: Regulation and Clinical Applications, S. Dagogo-Jack (ed.), Springer International Publishing Switzerland 2015).

In some embodiments, a loss of function mutation in an endogenous rodent Lepr gene comprises a point mutation in an exon of an endogenous rodent Lepr gene. In some embodiments, a loss of function mutation comprises a deletion, in whole or in part, of the coding sequence of an endogenous rodent Lepr gene. In some embodiments, the deletion comprises a nucleotide sequence of an endogenous rodent Lepr gene encoding the extracellular domain in whole or in part. In some embodiments, the deletion comprises exon 1, exon 2, exon 3, exon 4, exon 5, and/or exon 6, each in whole or in part, of an endogenous rodent Lepr gene. In some embodiments, a loss of function mutation comprises a deletion of the coding portion of exon 1 through exon 6 of an endogenous rodent Lepr gene, and an insertion of a reporter gene (e.g., LacZ).

In a further aspect, disclosed herein is use of a rodent whose genome comprises a loss of function mutation in an endogenous Slc39a5 gene as an animal model, which permits elucidation of the mechanisms of Slc39a5 action in the context of glycemic regulation and provides opportunities to test and develop therapeutics to target Slc39a5 in the treatment of metabolic and cardiovascular disorders.

In some embodiments, a rodent whose genome comprises a loss of function mutation in an endogenous Slc39a5 gene is used in a method of testing, screening, or identifying an agent that inhibits the activity of a Slc39a5 protein. In accordance with such method, a rodent whose genome comprises a loss of function mutation in an endogenous Slc39a5 gene is used along with a wild type rodent without the mutation, and a candidate agent is administered to the wild type rodent. Both the rodent with the loss of function mutation and the wild type rodent are examined to measure the serum zinc levels and one or more metabolic and cardiovascular traits, including one or more liver, lipid or glycemic traits. The measurements from the wild type rodent after the administration of the agent, from the wild type rodent before the administration (or from another wild type rodent not administered with the agent), and from the rodent with the loss of function mutation, are compared with one another to determine whether the agent inhibits the activity of a Slc39a5 protein. An agent that results in an elevated serum zinc level and an improvement in one or more traits in the same direction as the rodent with the loss of function mutation relative to the wild type rodent before the administration (or another wild type rodent not administered the agent) is considered to inhibit the activity of a Slc39a5 protein.

In some embodiments, a rodent homozygous for a loss of function mutation in an endogenous Slc39a5 gene is used. In some embodiments, a rodent heterozygous for a loss of function mutation in an endogenous Slc39a5 gene is used.

In some embodiments, the rodent having a loss of function mutation in an endogenous Slc39a5 gene is a female rodent. In some embodiments, the rodent having a loss of function mutation in an endogenous Slc39a5 gene is a male rodent.

In particular embodiments, the rodent having a loss of function mutation in an endogenous Slc39a5 gene is a female rodent (e.g., a mouse or a rat) homozygous for the loss of function mutation.

In some embodiments, a candidate agent is an antibody specific for a Slc39a5 protein (e.g., a human SLC39A5 protein).

As disclosed herein, both homozygous and heterozygous inactivation of an endogenous Slc39a5 gene result in elevation in serum zinc levels in rodent animals. An agent that results in an elevated serum zinc level and an improvement in one or more traits is considered as an agent that inhibits the activity of a Slc39a5 protein. In some embodiments, an agent results in an elevation in the serum zinc level in a wild type rodent administered with the agent by at least 10%, 11%, 12%, 13%, 14%, 15%, 20%, 25%, or more, relative to a wild type rodent not administered the agent.

In some embodiments, at least one of the traits measured is a glycemic trait, e.g., the fasting glucose level in the serum. It has been shown herein that female mice homozygous for Slc39a5 loss of function exhibit reduced fasting blood sugar levels as compared to littermate controls. Accordingly, an agent that results a reduction in the fasting glucose level in a wild type rodent administered with the agent relative to a wild type rodent not administered the agent is considered an agent that inhibits the activity of a Slc39a5 protein. In some embodiments, the reduction is by at least 5%, 6%, 7%, 8%, 9%, 10%, 11%, 12%, 13%, 14%, 15%, or more.

In some embodiments, at least one of the traits measured is a liver trait, e.g., the serum level of alanine aminotransferase and/or aspartate aminotransferase, or the extent of hepatic steatosis in rodents after being fed with a high fat diet, and in some embodiments, the high fat diet is a high fat high fructose diet. It has been shown herein that female mice homozygous for Slc39a5 loss of function exhibit reduced hepatic steatosis and improvements in serum ALT and AST levels on a high fat diet as compared to wild type rodents on a high fat diet. An agent that results a reduction in hepatic steatosis in a wild type rodent administered with the agent relative to a wild type rodent not administered the agent is considered an agent that inhibits the activity of a Slc39a5 protein. An agent that results in improvement (i.e., reduction) in serum ALT and/or AST levels in a wild type rodent on a high fat diet and administered the agent, as compared to a wild type rodent on a high fat diet not administered the agent, is considered an agent that inhibits the activity of a Slc39a5 protein. In some embodiments, the improvement (i.e., the reduction in the level of ALT and/or AST) is by at least 10%, 15%, 20%, 25%, 30%, 35%, 40%, 45%, 50%, or more.

In some embodiments, at least one of the traits measured is body weight. Both male and female mice homozygous for Slc39a5 loss of function have been shown herein to have a reduced body weight. An agent that results a reduction in body weight is considered an agent that inhibits the activity of a Slc39a5 protein. Agents that have been identified to inhibit the activity of a Slc39a5 protein may be used in the treatment of metabolic and cardiovascular disorders including, for example, increased serum glucose level, hyperglycemia, Type 2 diabetes, obesity, increased low density lipoprotein (LDL), decreased high density lipoprotein (HDL), alcoholic fatty-liver disease, non-alcoholic fatty liver disease, non-alcoholic steatohepatitis, and hepatic encephalopathy.

The present description is further illustrated by the following examples, which should not be construed as limiting in any way. The contents of all cited references (including literature references, issued patents, and published patent applications as cited throughout this application) are hereby expressly incorporated by reference.

### Example 1. Generation of Slc39a5 Loss of Function Mice

The genetically engineered Slc39a5^{-/-} mouse strain was created using Regeneron's VelociGene® technology (Valenzuela et al., Nat Biotechnol. 2003; 21(6):652-9; Poueymirou et al., Nat Biotechnol. 2007; 25(1):91-9). FIG. 3 depicts the strategy. Briefly, C57Bl/6NTac embryonic stem cells (ESC) were targeted for ablation of a portion of the Slc39a5 locus, beginning just after the start ATG codon and ending 5 base pairs before the 3′ end of coding exon 2. This region contains the Slc39a5 signal peptide and much of the N-terminal extracellular domain. A lacZ reporter module was inserted in frame with the Slc39a5 start, followed by a fLoxed neomycin resistance cassette for selection in ESC. The resistance cassette was deleted prior to microinjection using self-deleting technology. The targeted cells were microinjected into 8-cell embryos from Charles River Laboratories Swiss Webster albino mice, yielding F0 VelociMice® that were 100% derived from the targeted cells (Poueymirou et al. 2007). These mice were subsequently bred to homozygosity. Slc39a5^{-/-} heterozygous mice and C57Bl/6NTac wildtype littermates were used as indicated.

### Example 2. Metabolic Phenotyping of Slc39a5 Loss of Function Mice

Serum Zinc and Fasting Blood Glucose Levels of Slc39a5 Loss of Function Mice.

Mice deficient in Slc39a5 along with heterozygous and wild-type littermates were co-housed in a controlled environment (12 hr light/dark cycle, 22±1° C., 60-70% humidity) and fed ad-libitum with standard chow (PicoLab Rodent Diet 20, Catalog #5053) containing 87 ppm zinc. Both male and female mice were used in this study. Mice were monitored for growth kinetics by recording body weight twice a month. Upon an overnight fast (lasting 16 hours), blood was sampled via a submandibular incision when the mice were 8 weeks of age. Serum zinc was measured using flame atomic absorption spectroscopy as described previously (Prasad et al., J Lab Clin. Med. 1963; 61: 537-49) and fasting blood glucose was evaluated using AlphaTrak blood glucose monitoring system (Zoetis United States, Parsippany N.J.).

Hepatic Function of Slc39a5 Loss of Function Mice on Long-Term High Fat Diet (HFD).

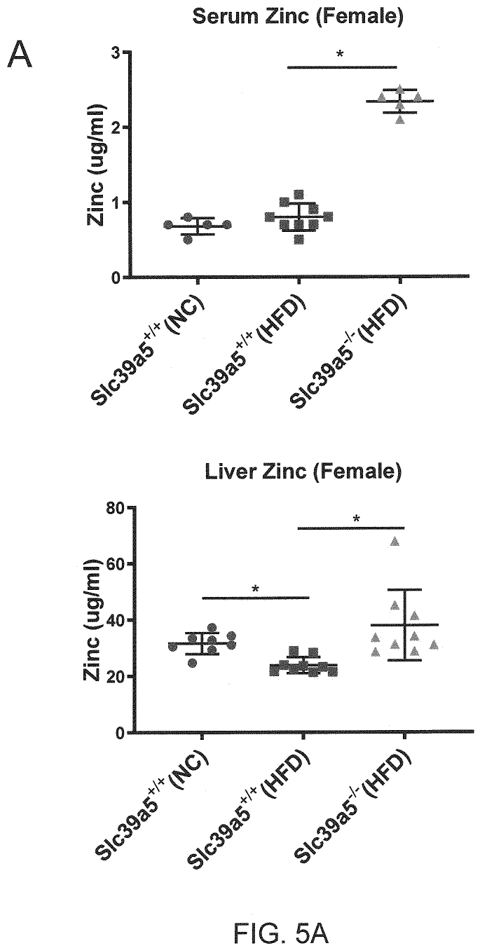

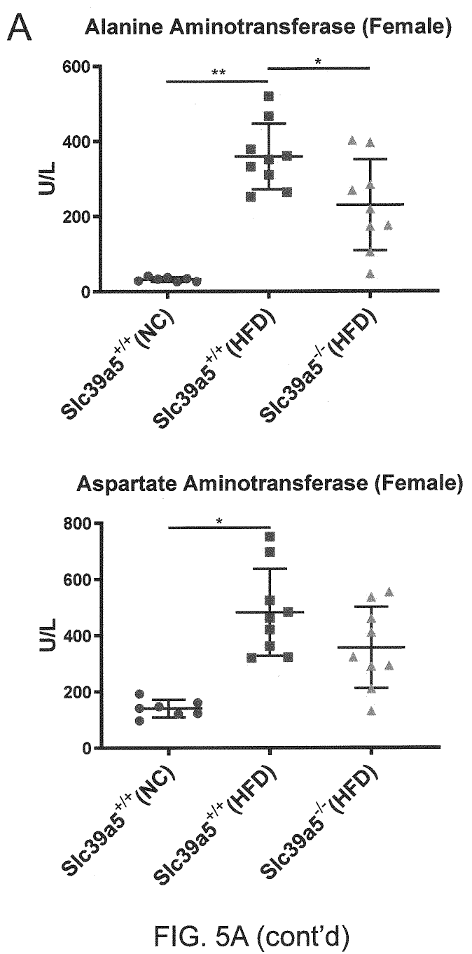

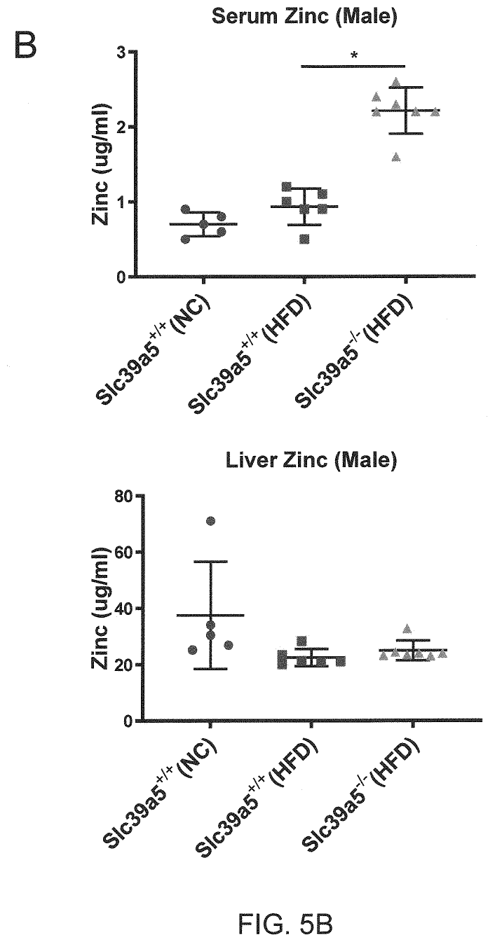

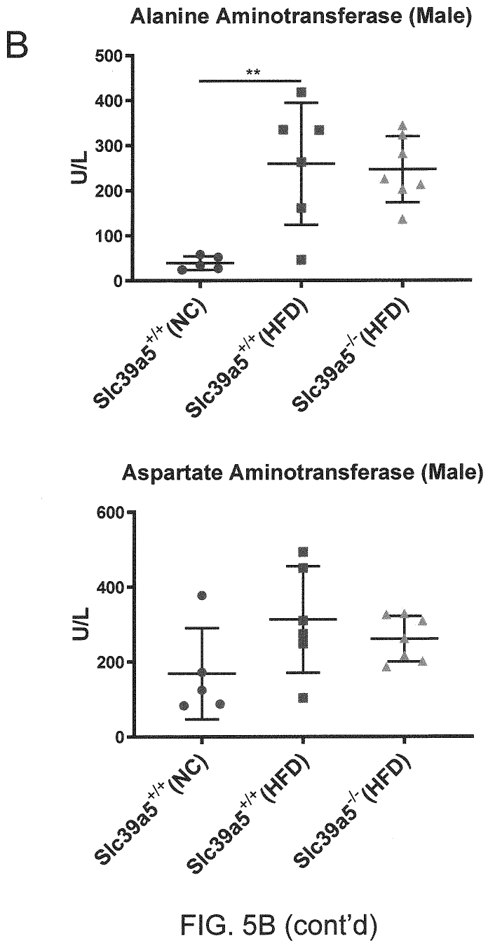

Mice homozygous for Slc39a5 loss of function and wild-type littermates were co-housed in a controlled environment (12 hr light/dark cycle, 22±1° C., 60-70% humidity) and fed ad-libitum with a high fat diet (Test Diet, Catalog #9GWP) containing 35 ppm zinc starting at 6 weeks of age. Both male and female mice were used in this study. Upon an overnight fast (lasting 16 hours), levels of serum and hepatic zinc along with ALT and AST (biomarkers of liver injury) were assessed at 40 weeks of high fat diet challenge (FIGS. 5A-B). A separate cohort of age matched wild-type C57BLK/6 mice maintained on normal chow (Lab Diet, Catalog #5K52) containing 85 ppm zinc were obtained from Jackson Laboratories as controls. Levels of serum and hepatic zinc were measured using flame atomic absorption spectroscopy as discussed earlier. Serum ALT and AST levels were measured using ADVIA Chemistry XPT System (Siemens Healthineers). Explanted liver samples were fixed in 10% phosphate buffered formalin acetate at 4° C. overnight, thoroughly rinsed in phosphate-buffered saline and embedded in paraffin wax. Hematoxylin and eosin staining was performed on 5 μm thick paraffin sections using standard histochemistry techniques. Sections were imaged using a 40× objective using the EVOS FL Auto microscope (Thermo Fisher Scientific).

Liver, Lipid and Glycemic Traits of Mice Homozygous for Slc39a5 and Leptin-Receptor (Lepr) Loss-of-Function.

Female mice homozygous for Slc39a5 and Lepr loss of function and littermate controls (wild-type, Slc39a5^{-/-}, Lepr^{-/-}) were co-housed in a controlled environment (12 hr light/dark cycle, 22±1° C., 60-70% humidity) and fed ad-libitum with normal chow (PicoLab Rodent Diet 20, Catalog #5053) containing 87 ppm zinc. Mice were monitored for health and growth kinetics periodically. Upon an overnight fast (lasting 16 hours), levels of serum ALT and AST (biomarkers of liver injury) along with LDL-C and fasting blood glucose were measured when the mice were 22 weeks of age. An oral glucose tolerance test was conducted by administering 2 g/kg of body weight of Dextrose (Hospira Inc. NDC 0409-4902-34) by oral gavage upon an overnight fast (lasting 16 hours) at 22 weeks of age. Blood glucose was evaluated at defined time points (0, 15, 30, 60 and 120 minutes) using AlphaTrak blood glucose monitoring system (Zoetis United States, Parsippany N.J.) by sampling blood from the lateral tail vein.

Data Analyses.

Data are reported as mean±SEM. Statistical analyses were performed using Prism 6.0 (GraphPad Software). All parameters were analyzed by two-way ANOVA or Student's t-test. *p<0.5, **p<0.01.

Results.

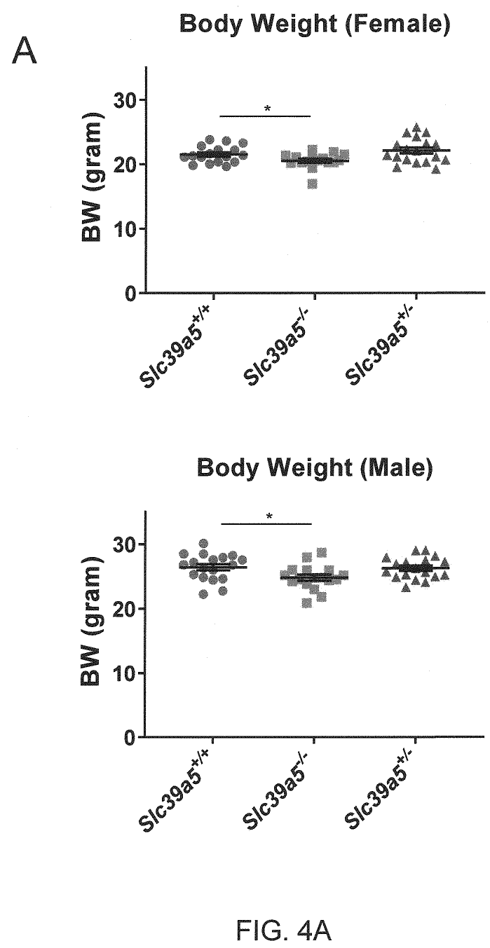

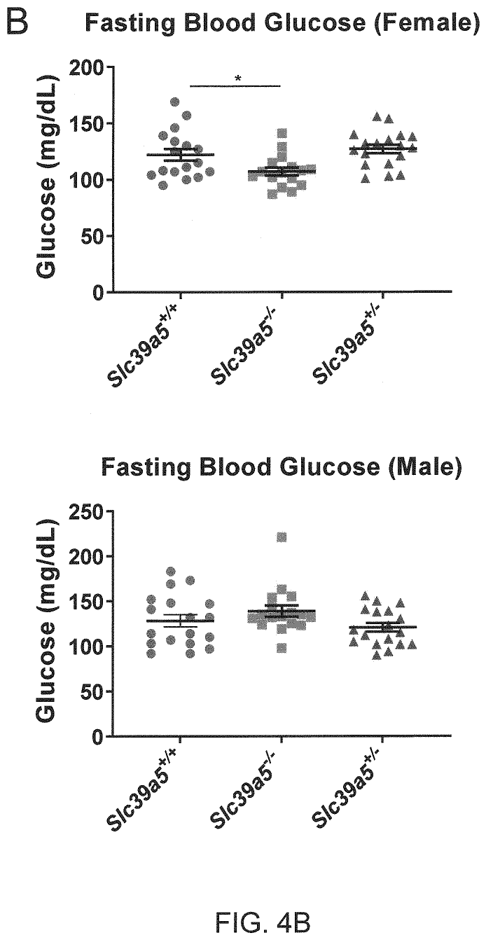

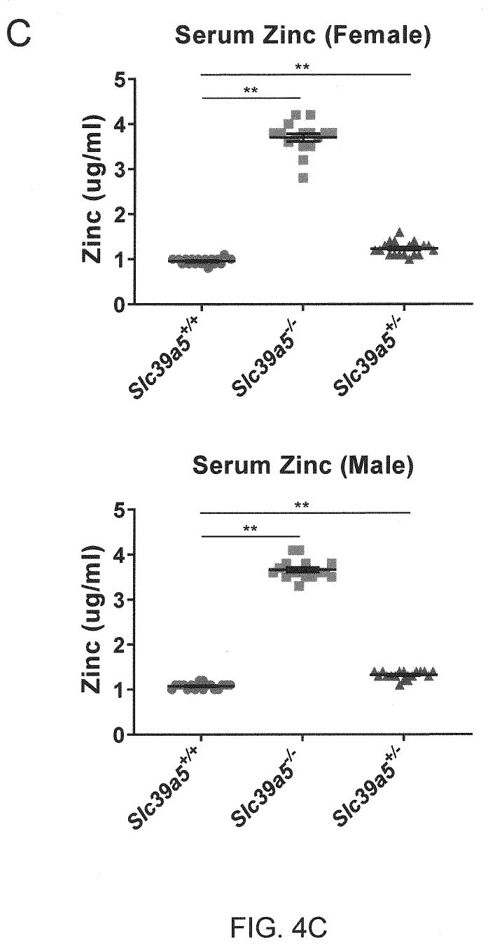

Mice homozygous for Slc39a5 loss of function had reduced body weight as compared to wild-type and heterozygous littermates at 8 weeks of age (FIG. 4A). Furthermore, regardless of sex, at 8 weeks of age mice homozygous for Slc39a5 loss of function had significantly elevated serum zinc levels as compared to wild-type and heterozygote littermates (FIG. 4C). Interestingly, female mice homozygous for Slc39a5 loss of function had reduced fasting blood sugar as compared to littermate controls (FIG. 4B, left panel). No differences were observed in male mice at this age.

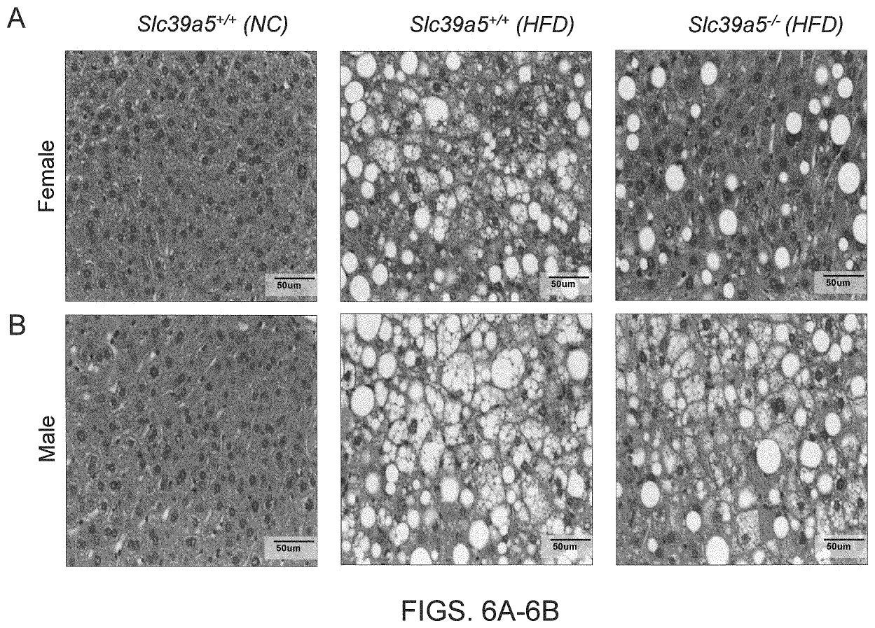

To study the apparent sexual dimorphism in fasting blood glucose levels, mice homozygous for Slc39a5 loss of function (male and female) were challenged with a high-fat diet regimen (60% kcal from fat) for 40 weeks and compared to wild-type mice on either high-fat diet or normal chow. Despite similar increases in serum zinc levels (FIGS. 5A-B, left most panel), only female mice showed increases in hepatic zinc levels and improvements in serum ALT and AST levels (biomarkers of liver injury) as compared to wild-type mice on high-fat diet. Consistent with the serum chemistry data, qualitative histochemical analyses of explanted liver samples demonstrated reduced hepatic steatosis in female mice homozygous for Slc39a5 loss of function as compared to wild-type counterparts on high fat diet. (FIG. 6A). No differences were observed between male mice homozygous for Slc39a5 loss of function and wild-type counterparts on high-fat diet (FIG. 6B, middle and right panels).

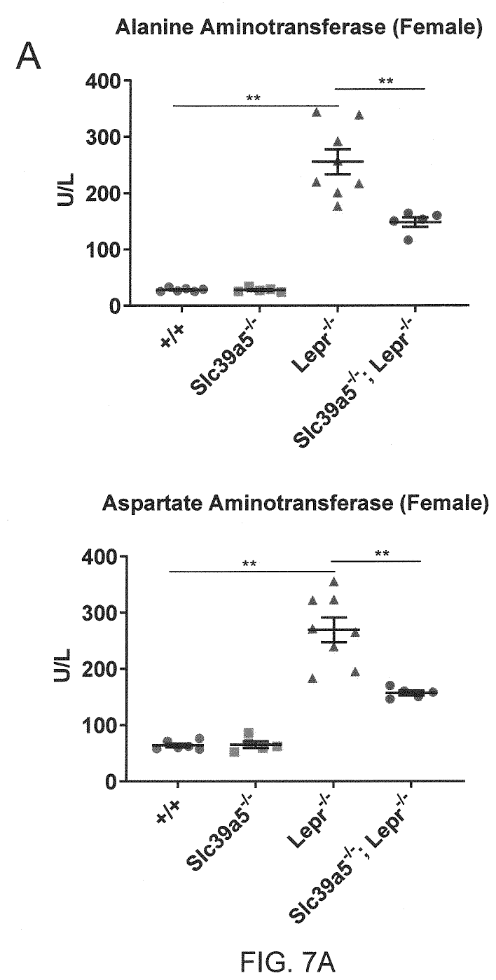

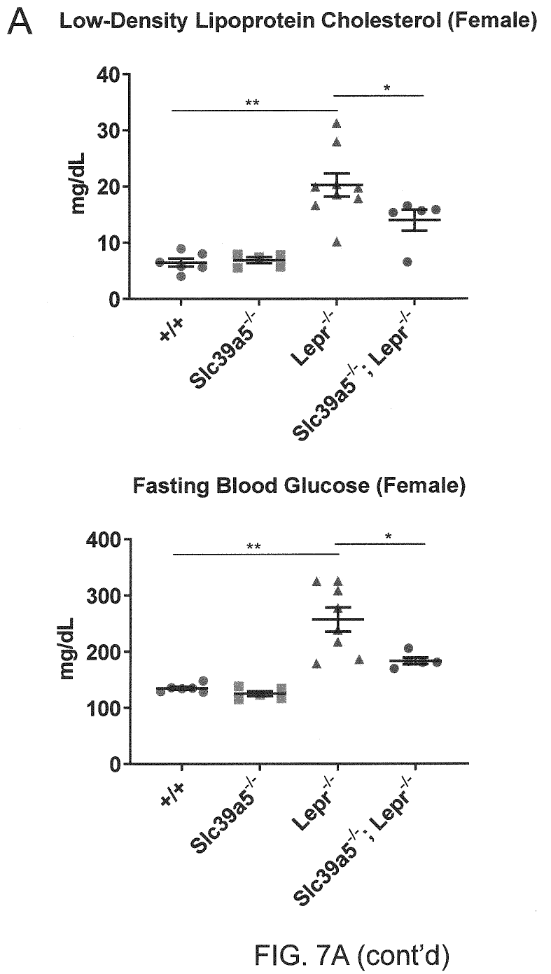

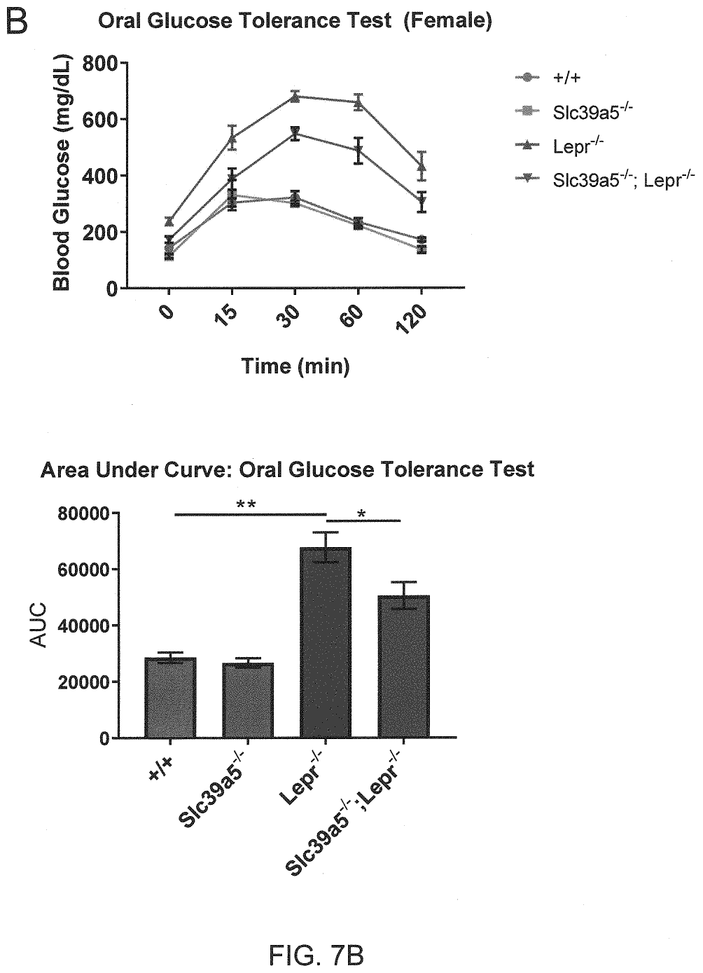

To further delineate this phenotype, in concurrent experiments female mice homozygous for Slc39a5 loss of function were bred into a leptin-receptor deficient background (a commonly used rodent model of obesity induced hyperglycemia and type II diabetes). As expected, leptin-receptor deficient mice were hyperglycemic and displayed elevated serum LDL-C levels (FIG. 7A, lower panel). Furthermore, these mice had elevated serum ALT and AST levels suggesting impaired liver function at 22 weeks of age. Loss of Slc39a5 function in female Lepr receptor deficient mice significantly reduced fasting blood glucose, serum LDL-C, serum ALT and AST levels potentially ameliorating the obesity induced metabolic dysregulation in leptin-receptor deficient mice (FIG. 7A). In line with these observations, female mice homozygous for Slc39a5 and Lepr loss of function demonstrated improved glucose tolerance when challenged with an oral glucose load (FIG. 7B).

### Example 3. Additional Metabolic Phenotyping of Slc39a5 Loss of Function Mice

### Metabolic Phenotyping:

Mice homozygous or heterozygous for Slc39a5 loss of function and wild-type littermates were co-housed in a controlled environment (12 hr light/dark cycle, 22±1° C., 60-70% humidity) and fed ad-libitum with a high fat high fructose diet (Test Diet, Catalog #5WK9) or a control diet (Test Diet, Catalog #58Y2) containing 35 ppm zinc starting at 10 weeks of age. Both male and female mice were used in this study. Longitudinal assessment of serum zinc, fasting blood glucose along with alanine aminotransferase (ALT) and aspartate aminotransferase (AST) (biomarkers of liver injury) were assessed upon an overnight fast (lasting 16 hours). Fed blood glucose was measured prior to the initiation of the fast. Serum and hepatic zinc (at endpoint) analyses were conducted using flame atomic absorption spectroscopy as discussed below.

Mice homozygous for Slc39a5 and Lepr loss of function (Slc39a5^{-/-}; Lepr^{-/-}) and littermate controls (wildtype, Slc39a5^{-/-}, Lepr^{-/-}) were co-housed in a controlled environment (12 hr light/dark cycle, 22±1° C., 60-70% humidity) and fed ad-libitum with normal chow (PicoLab Rodent Diet 20, Catalog #5053) containing 87 ppm zinc. Mice were monitored for health and growth kinetics periodically. Upon an overnight fast (lasting 16 hours), serum ALT and AST (biomarkers of liver injury) along with DLDL and fasting blood glucose were measured when the mice were 22 weeks of age. Blood glucose was evaluated using AlphaTrak blood glucose monitoring system (Zoetis United States, Parsippany N.J.) by sampling blood from the lateral tail vein. Liver and lipid traits were measured using a Siemens ADVIA Chemistry XPT as described below.

### Oral Glucose Tolerance Test:

An oral glucose tolerance test was administered upon an overnight fast (lasting 16 hours) at 20 weeks of age by administering 2 g/kg of body weight of Dextrose (Hospira Inc, NDC 0409-4902-34) by oral gavage. Blood glucose was evaluated at defined time points (0, 15, 30, 60 and 120 minutes) using AlphaTrak blood glucose monitoring system (Zoetis United States, Parsippany N.J.) by sampling blood from the lateral tail vein.

### Liver and Lipid Traits:

All liver and lipid traits were measured using ADVIA Chemistry XPT System (Siemens Healthineers), an FDA approved clinical analyzer which we maintain and operate according to Siemens' guidelines. The liver and lipid profile contains the following reagents: Alanine Aminotransferase (ALT, Siemens REF 03036926), Aspartate Aminotransferase (AST, Siemens REF 07499718), Cholesterol (CHOL, Siemens REF 10376501), Direct HDL Cholesterol (DHDL, Siemens REF 07511947), LDL Cholesterol Direct (DLDL, Siemens REF 09793248), Non-Esterified Fatty Acids (NEFA, Wako 999-34691, 995-34791, 991-34891, 993-35191), Triglycerides (TRIG, Siemens REF 10335892). These reagents when mixed with sample underwent redox reactions specific to the analyte of interest that bring about a color change proportional to the concentration of the analyte (colorimetric assay). Absorbance of light, in wavelength specific to the analyte, (from a Halogen light source) was measured and concentration determined. Each set of reagents was calibrated as recommended by manufacture and samples with known values (Multilevel Quality Controls) are measured daily. Parameters were never allowed to deviate from known means by more than one standard deviation. Samples were usually assayed undiluted, though they can be diluted up to 1.5× without affecting results. Samples were loaded into the analyzer in 0.6 ml microcentrifuge tubes and all reagent mixing, assay timing, absorbance and concentration calculation was performed by the analyzer.

### Metal Ion Quantification:

All ion measurements were performed using an Agilent Technologies 240 FS Atomic Absorption Spectrometer, in flame mode. Serum samples were quantitatively diluted in deionized water and subsequently analyzed. For the serum samples a Seronorm Trace Elements Serum (L-2) was used as reference. Tissue, bone and other material were first digested in nitric acid. The samples were weighed and incubated overnight at 85° C. in nitric. The following day, the samples were cooled down to room temperature and quantitatively transferred to polystyrene tubes with deionized water. Subsequently they were analyzed. For all tissue samples, a bovine liver standard reference material (SRM 1577c) from the National Institute of Standards and Technology was used as reference.

### Liver Histology and Immunoblotting:

Explanted liver samples were fixed in 10% phosphate buffered formalin acetate at 4° C. overnight, thoroughly rinsed in phosphate-buffered saline and embedded in paraffin wax. For hematoxylin and eosin staining, unstained 5 μm thick paraffin sections were deparaffinized in xylene then hydrated through graded alcohols up to water. Sections were stained with Carazzi's hematoxylin, washed in tap water, and then put in 95% ethanol. From there, they were put in eosin-phloxine solution then ran through graded alcohols to xylene. After xylene, the stained slides were cover-slipped and imaged stained and imaged using a 20× or 40× objective using the Aperio AT2 slide scanner (Leica Biosystems Inc.).

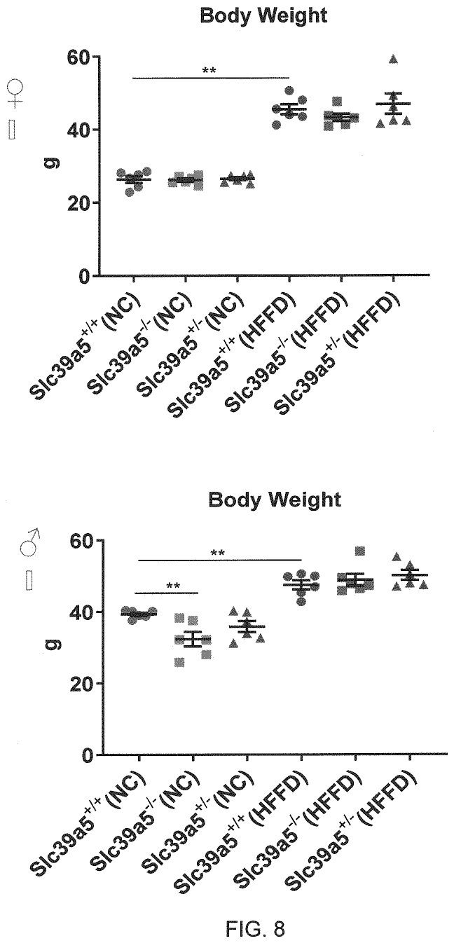

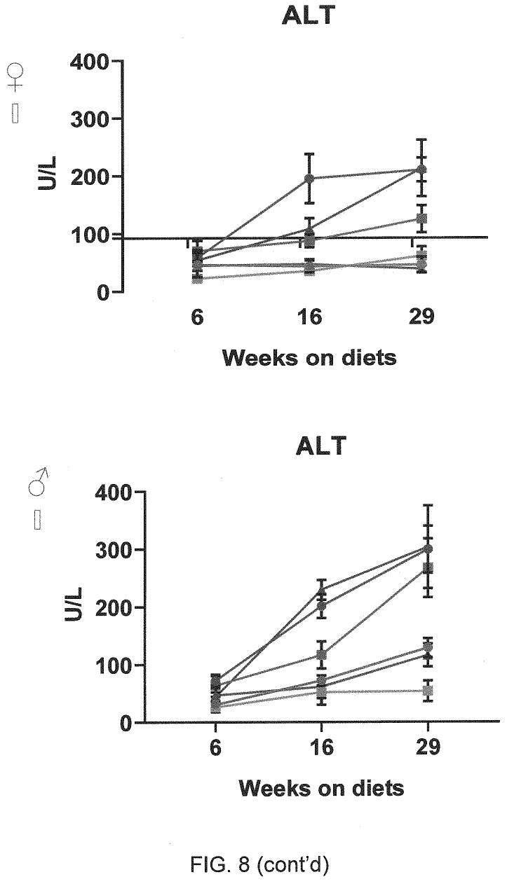

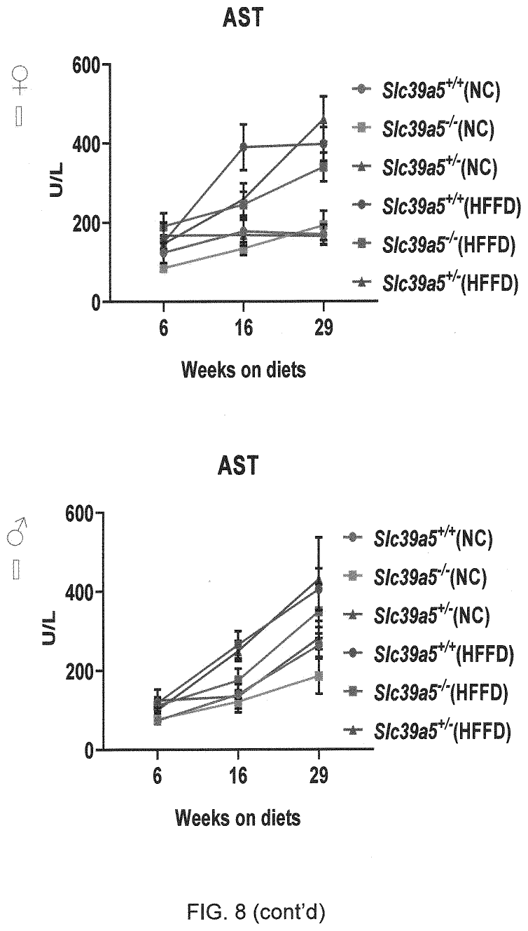

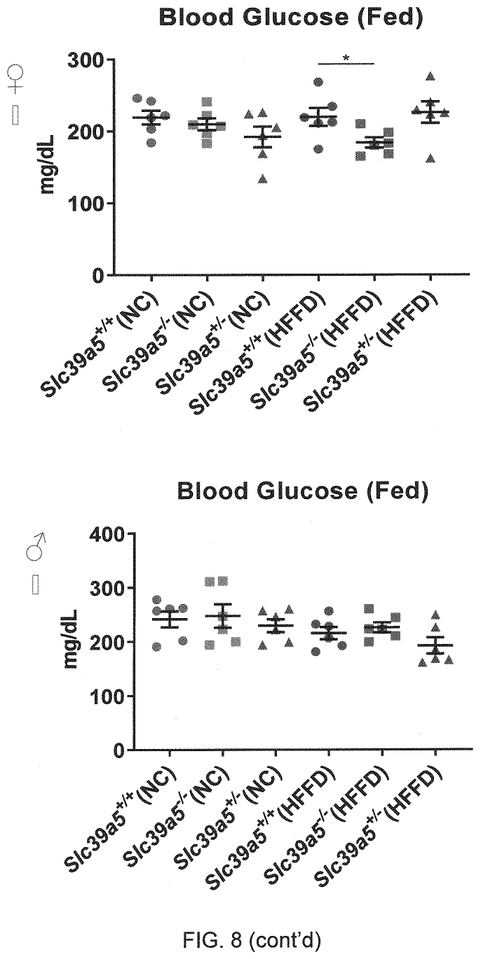

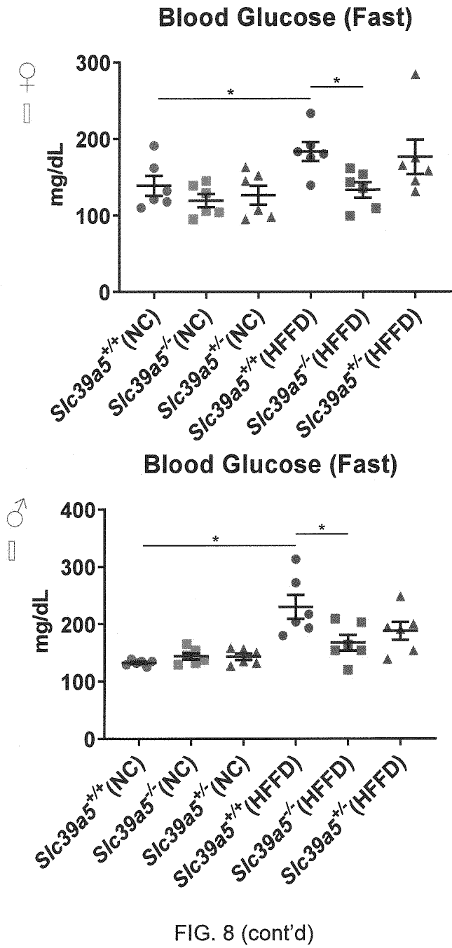

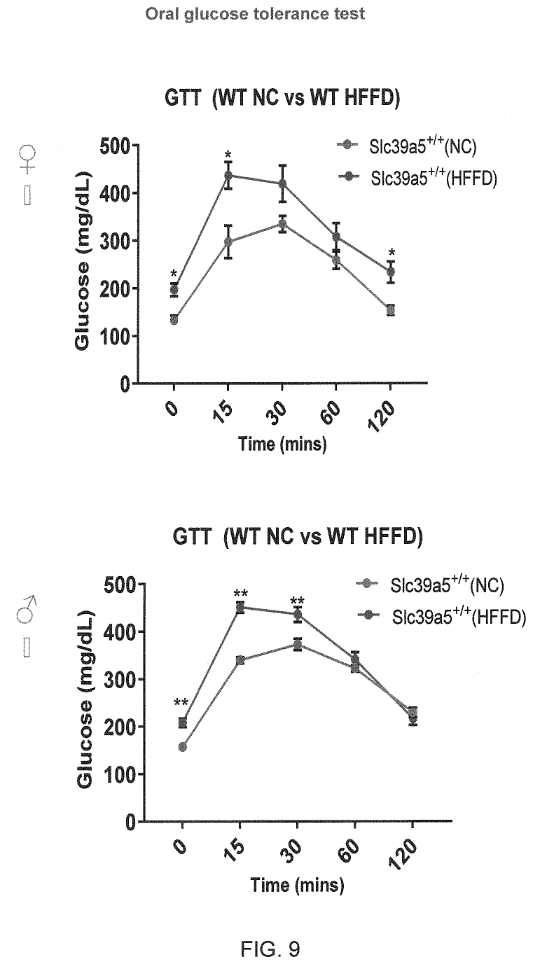

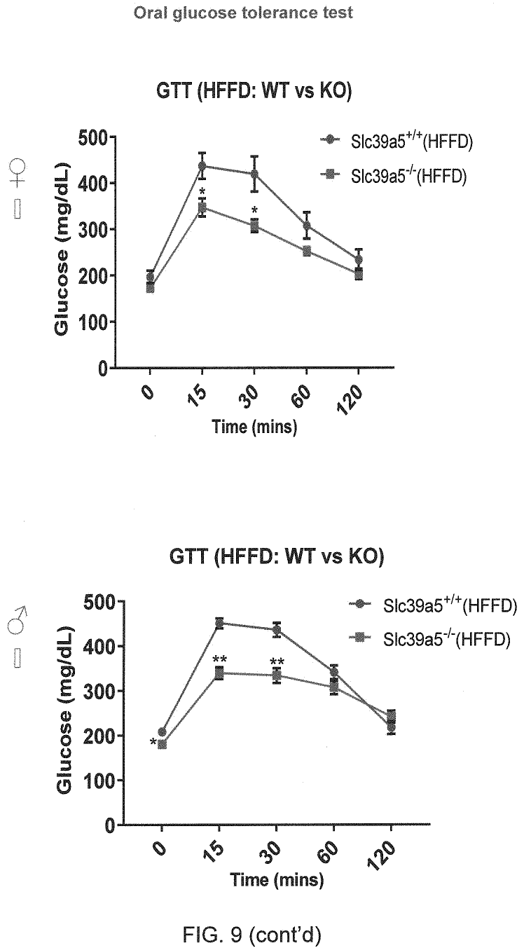

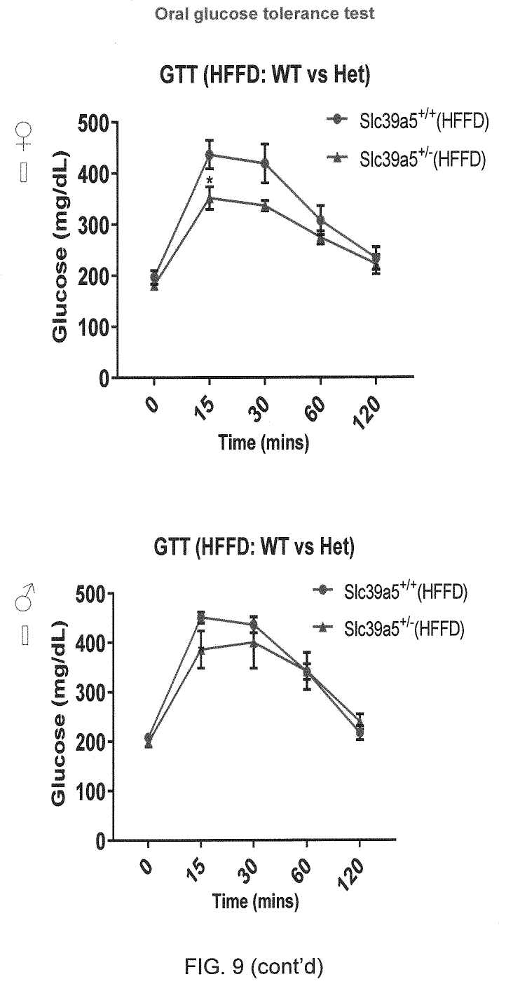

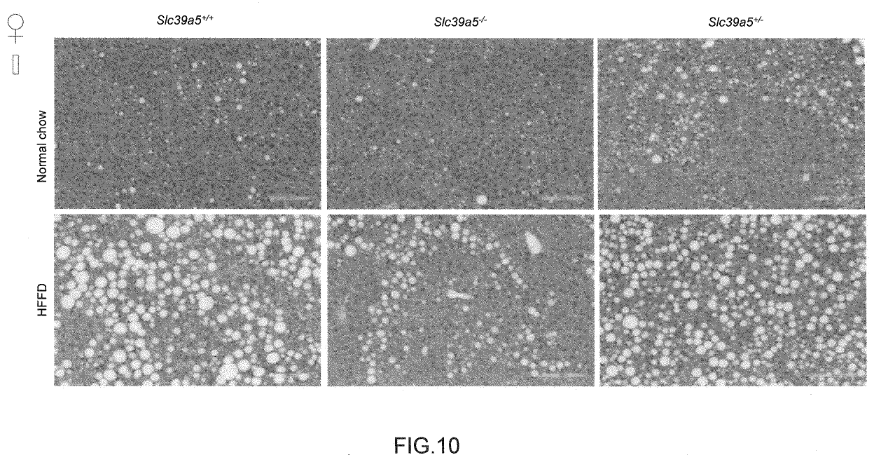

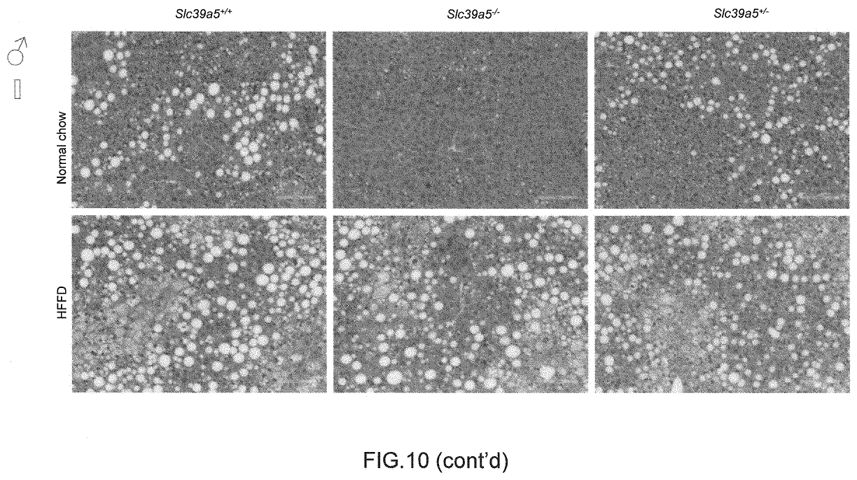

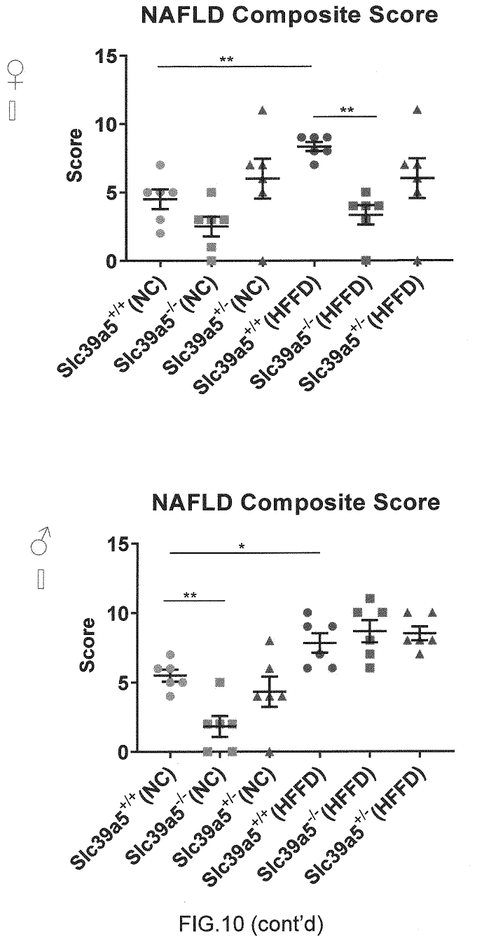

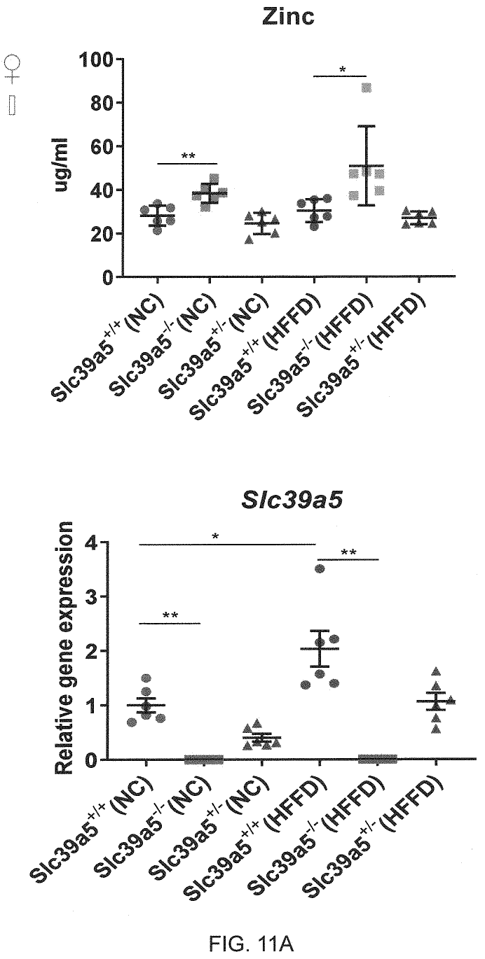

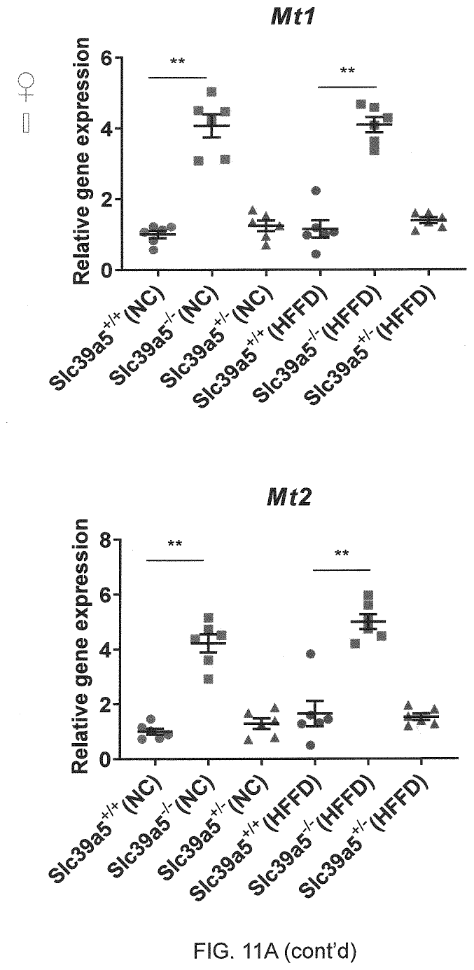

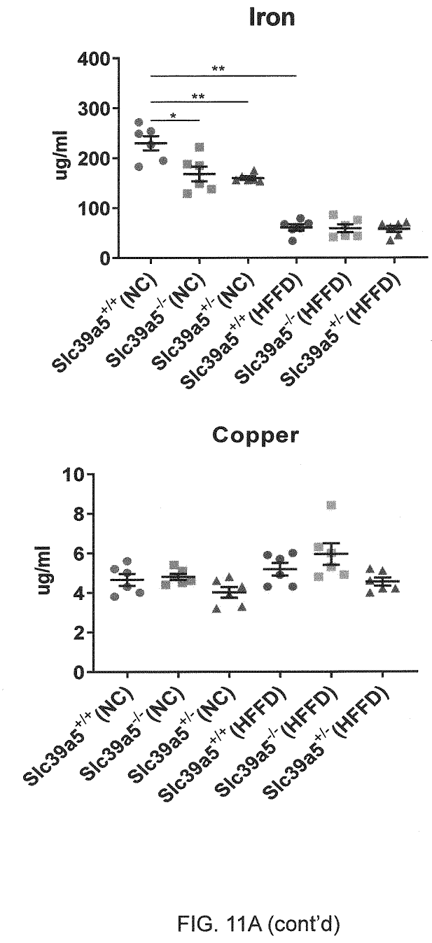

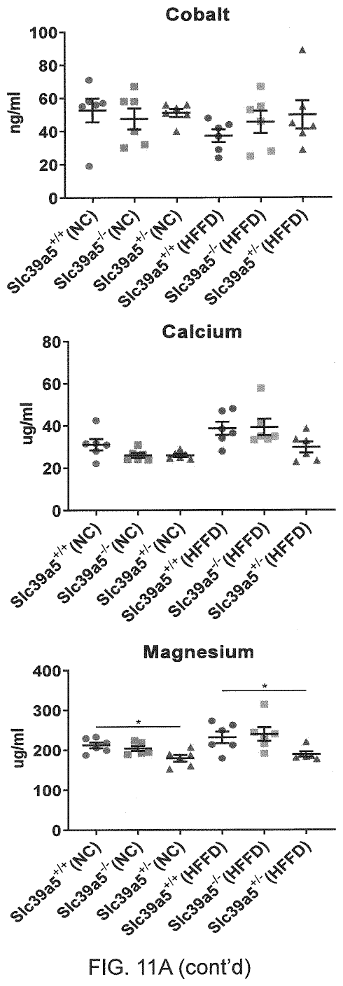

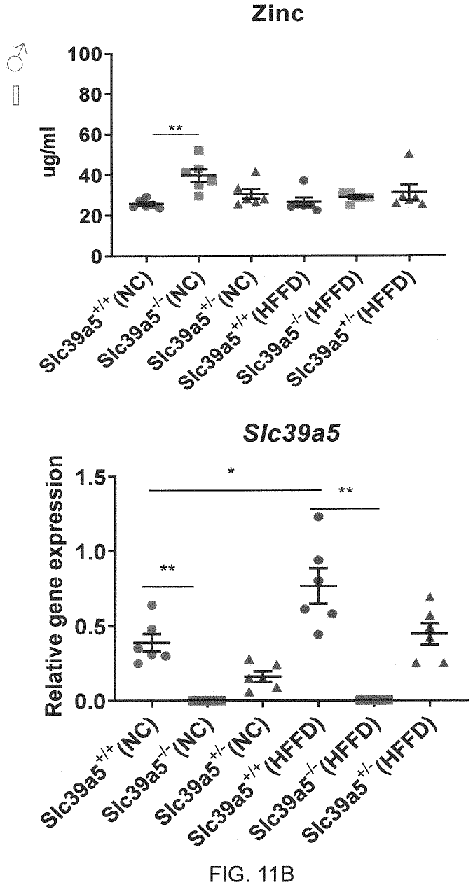

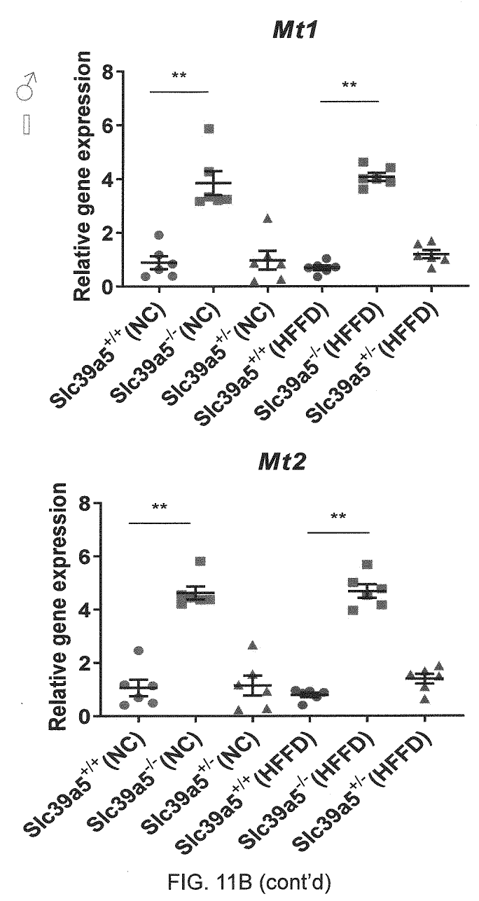

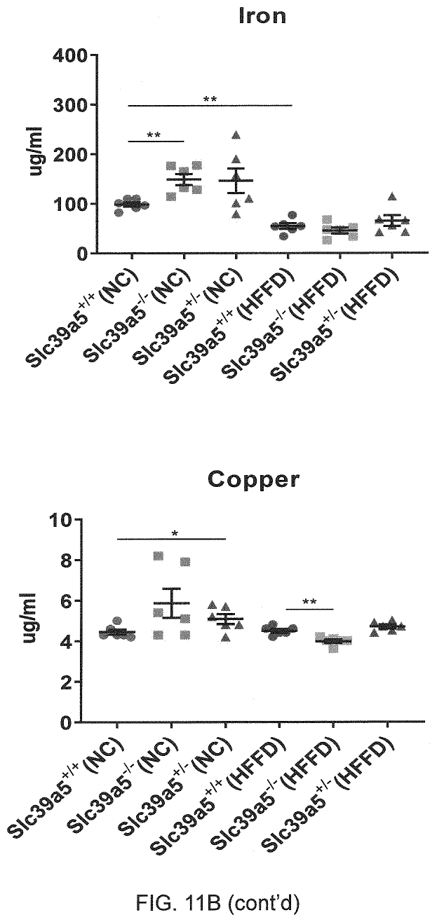

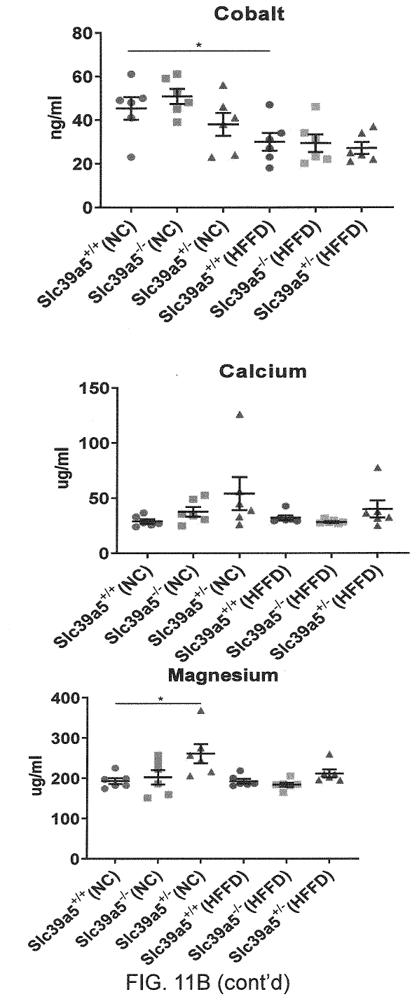

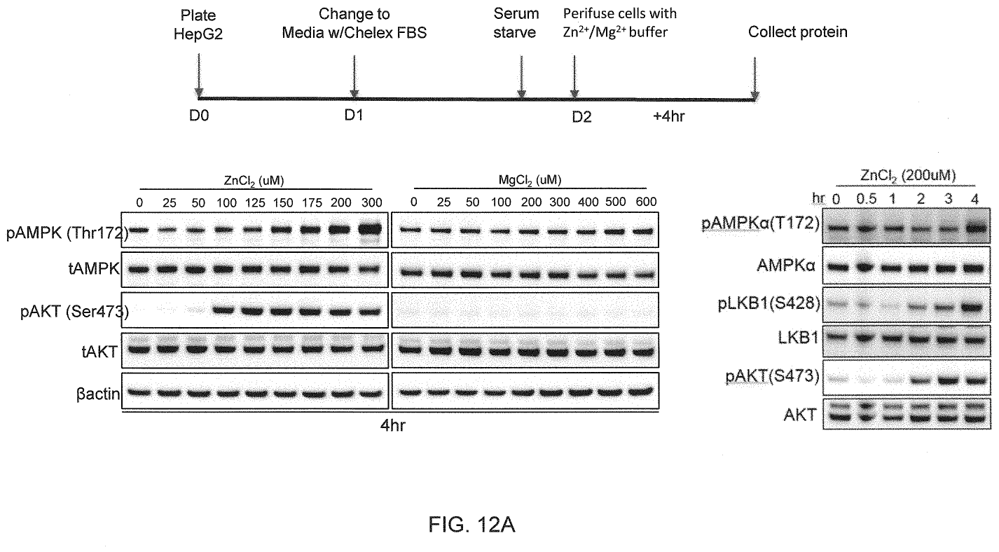

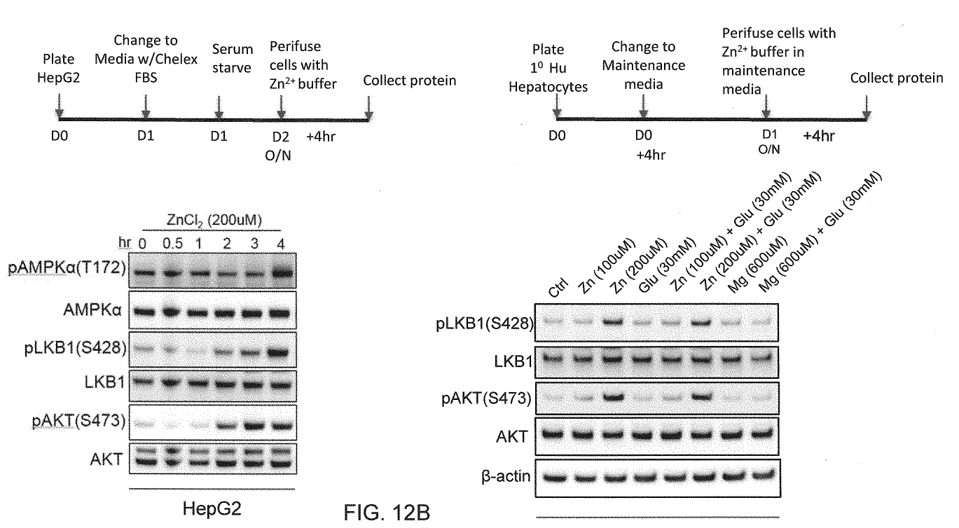

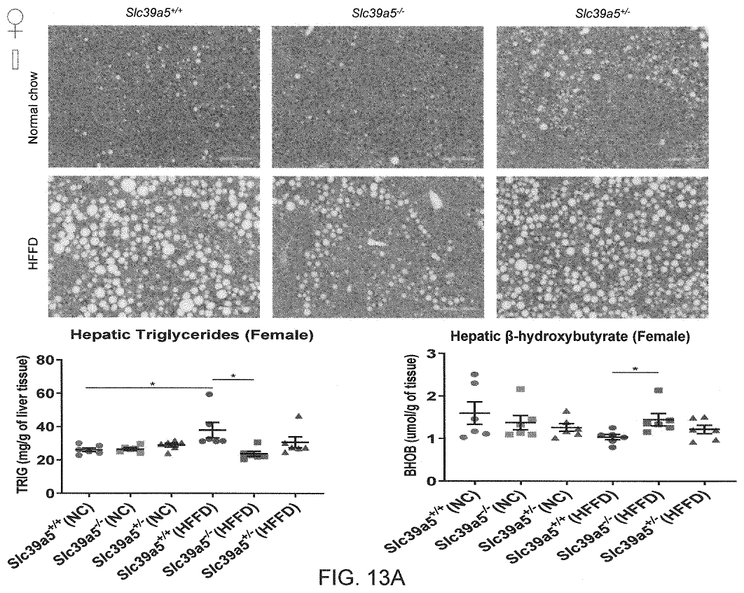

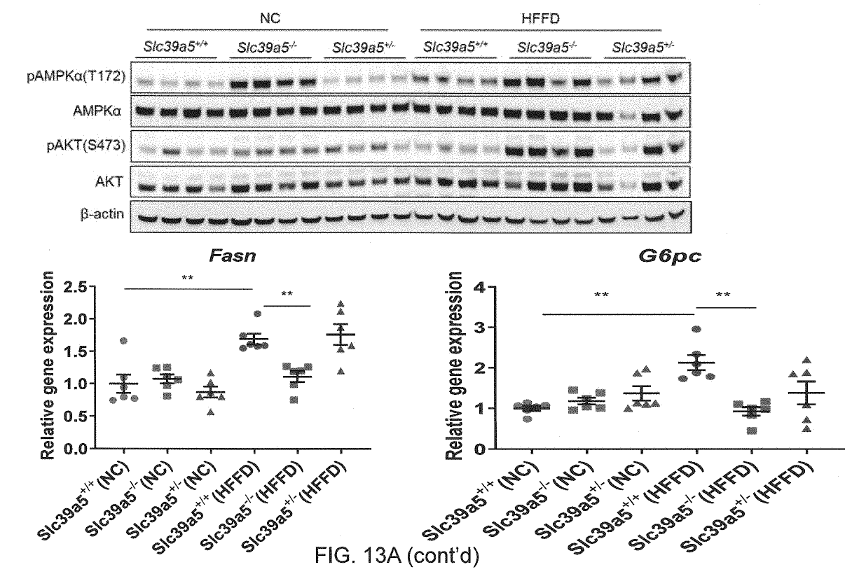

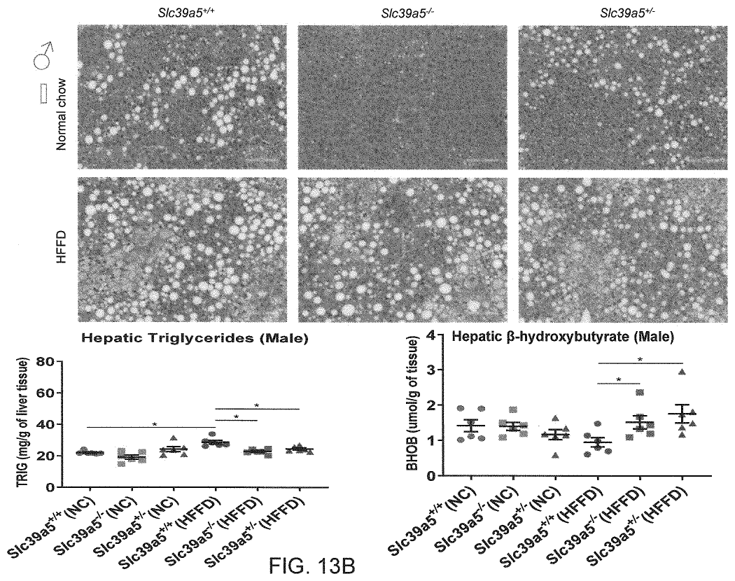

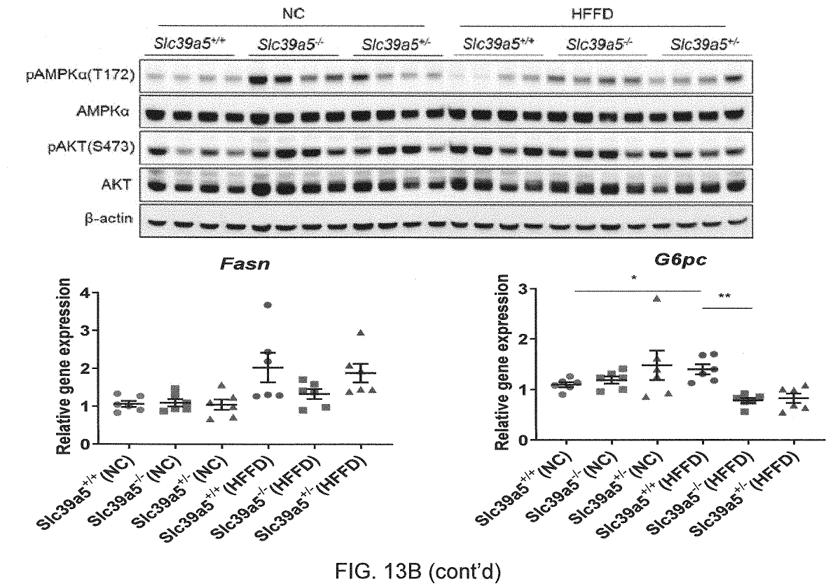

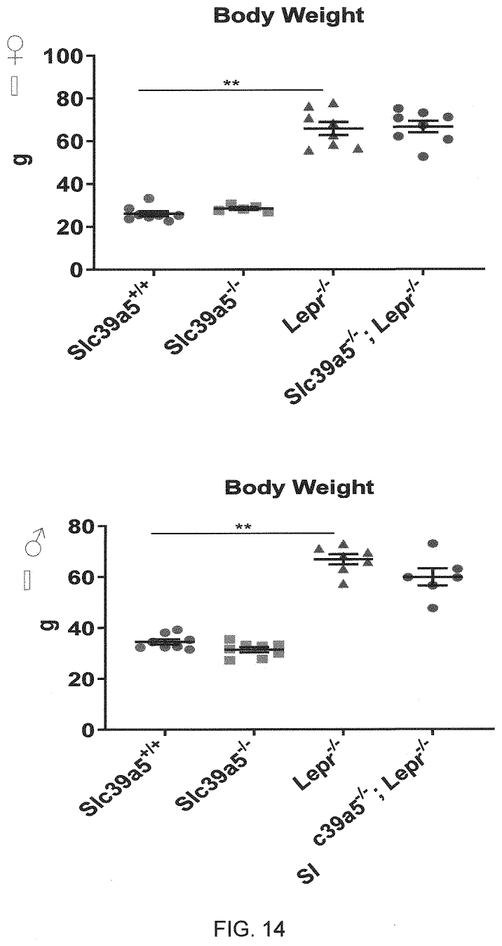

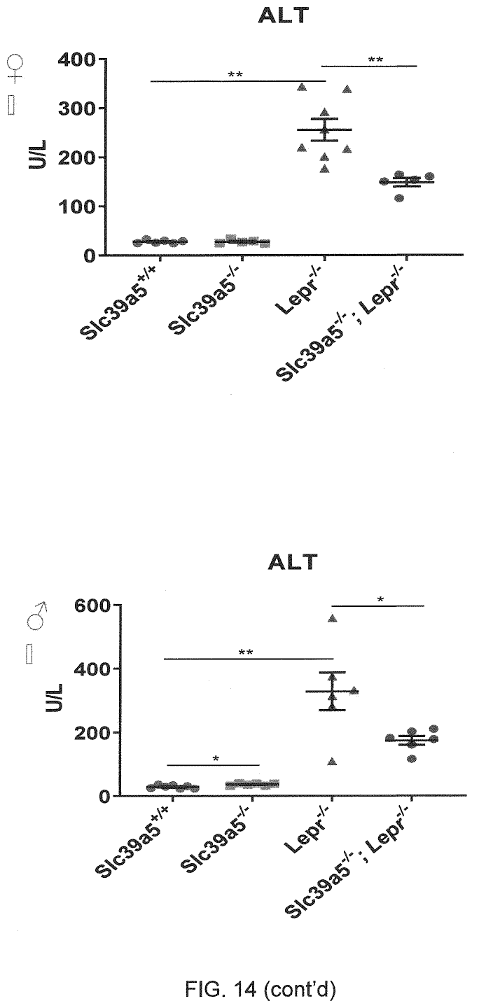

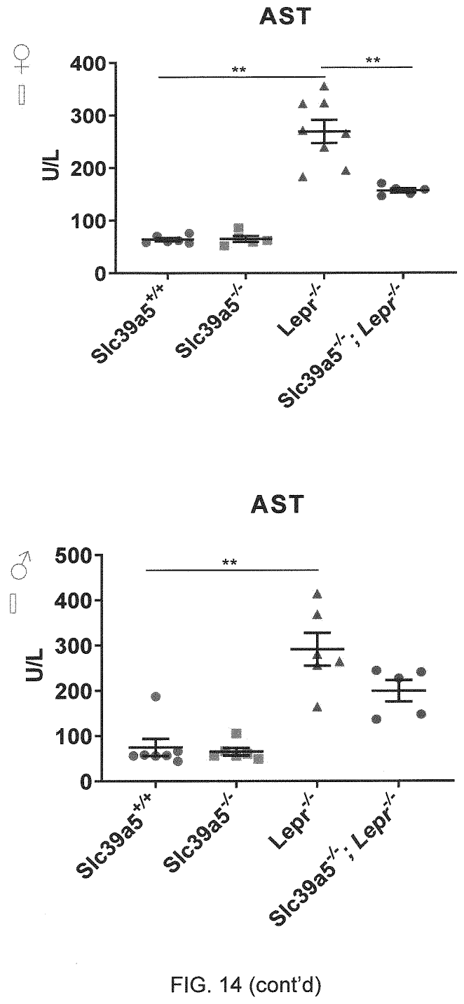

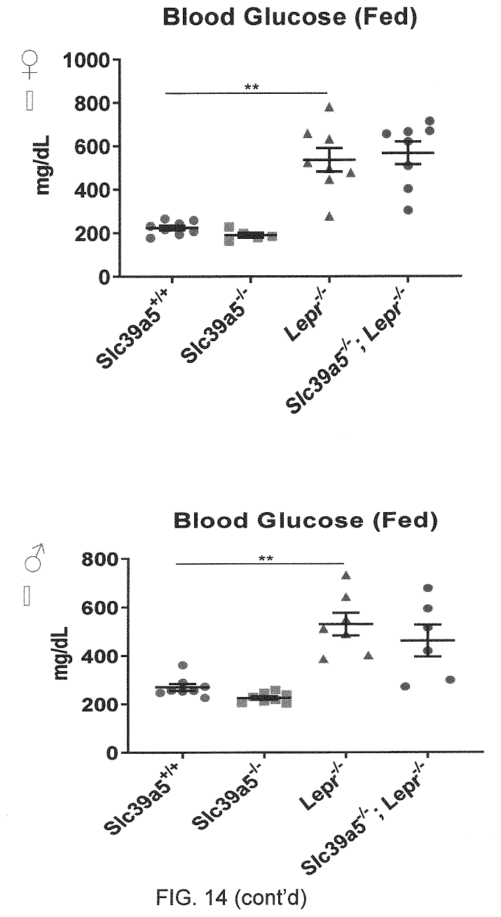

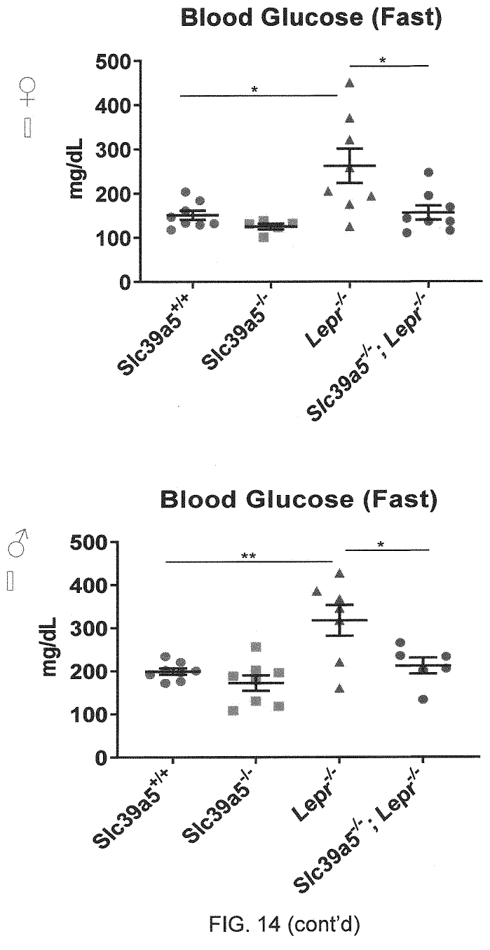

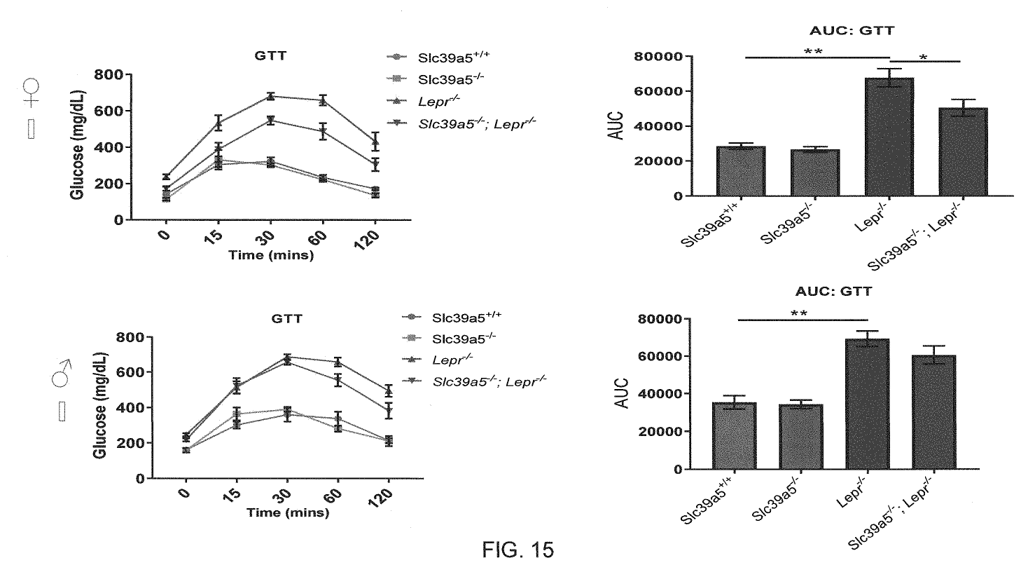

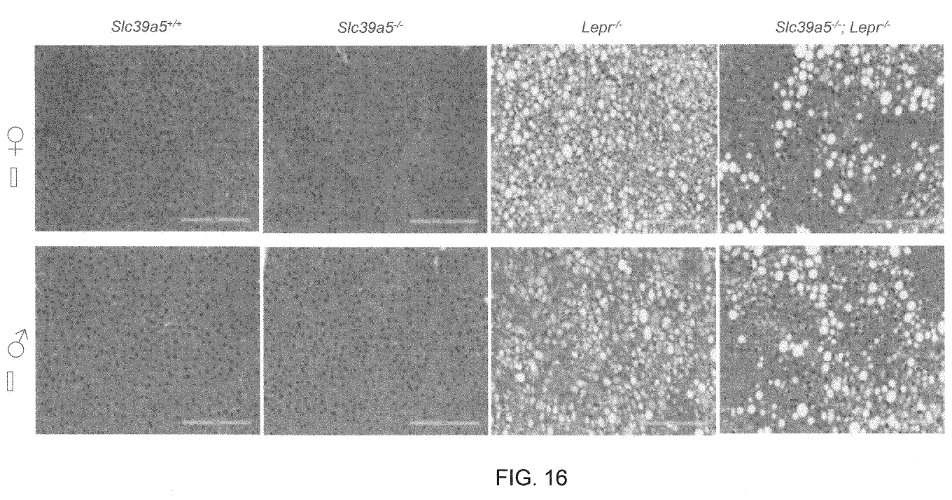

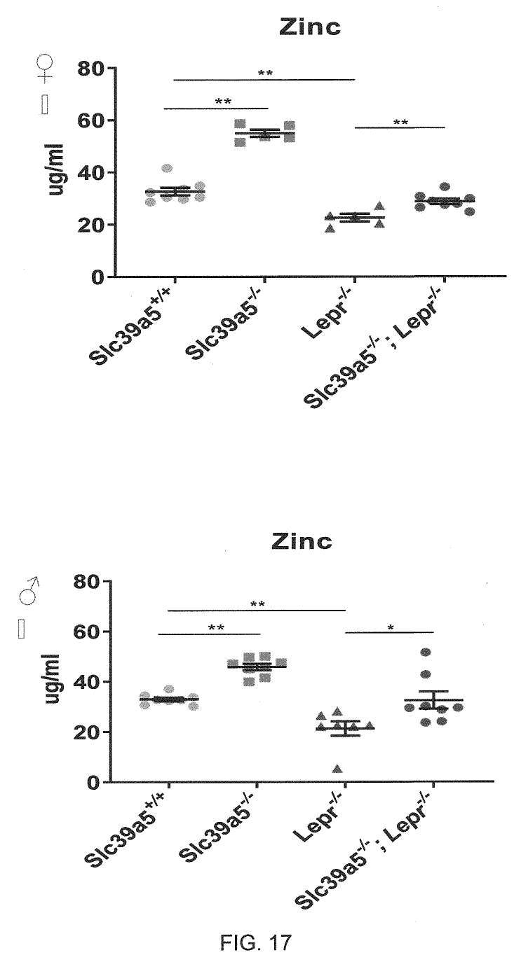

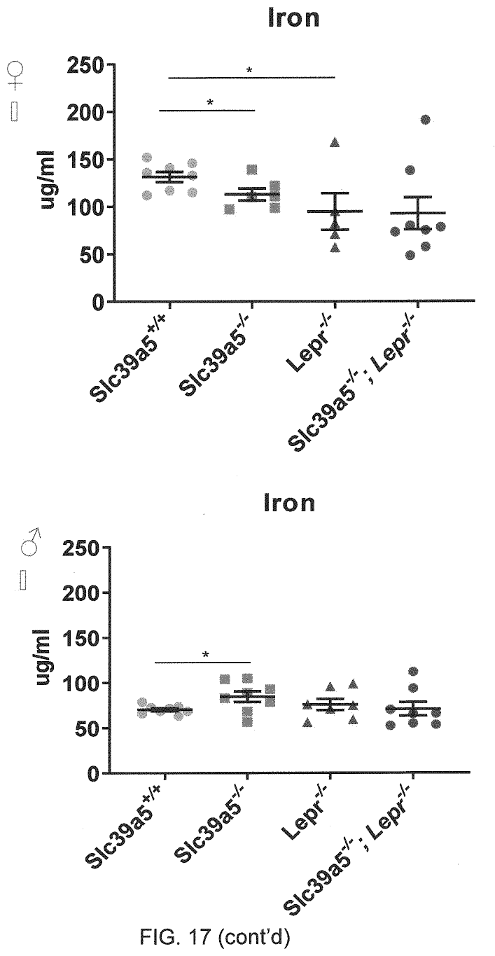

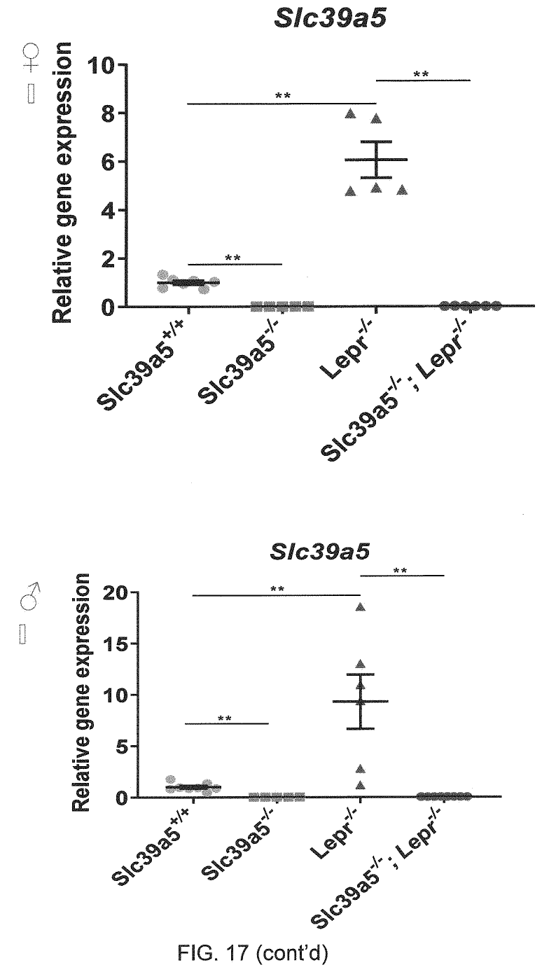

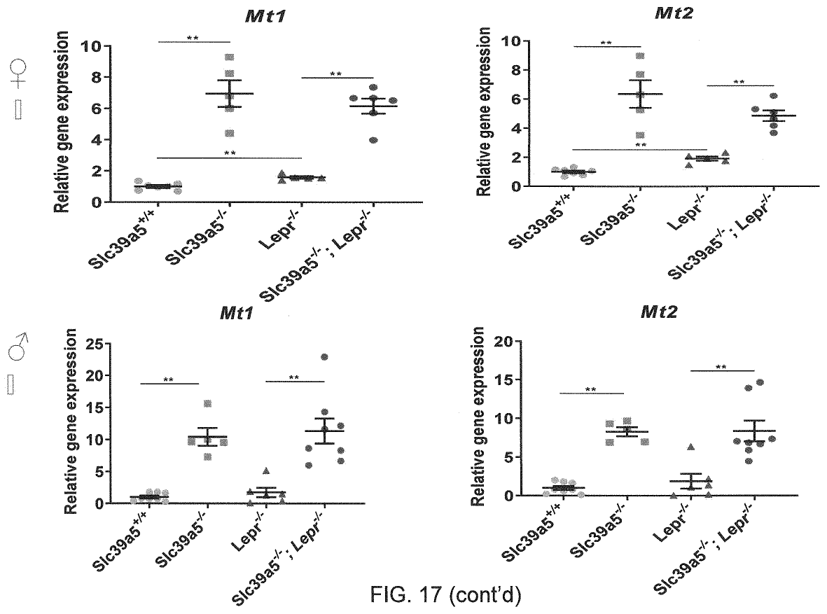

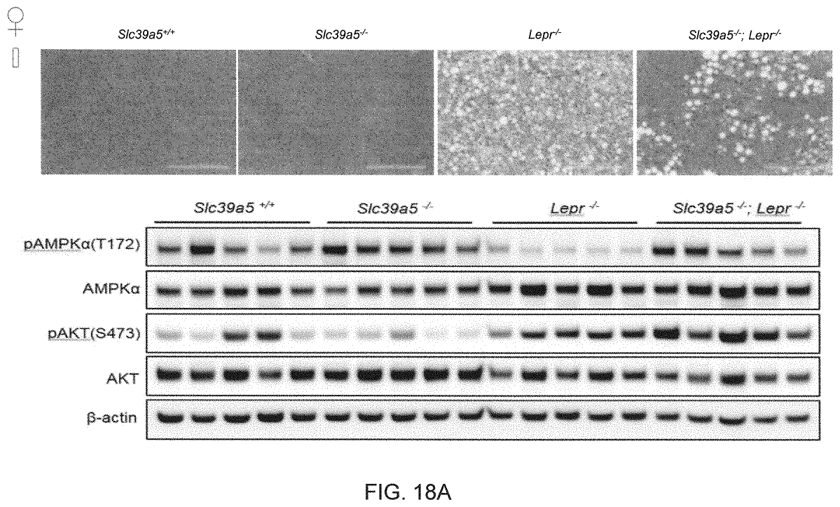

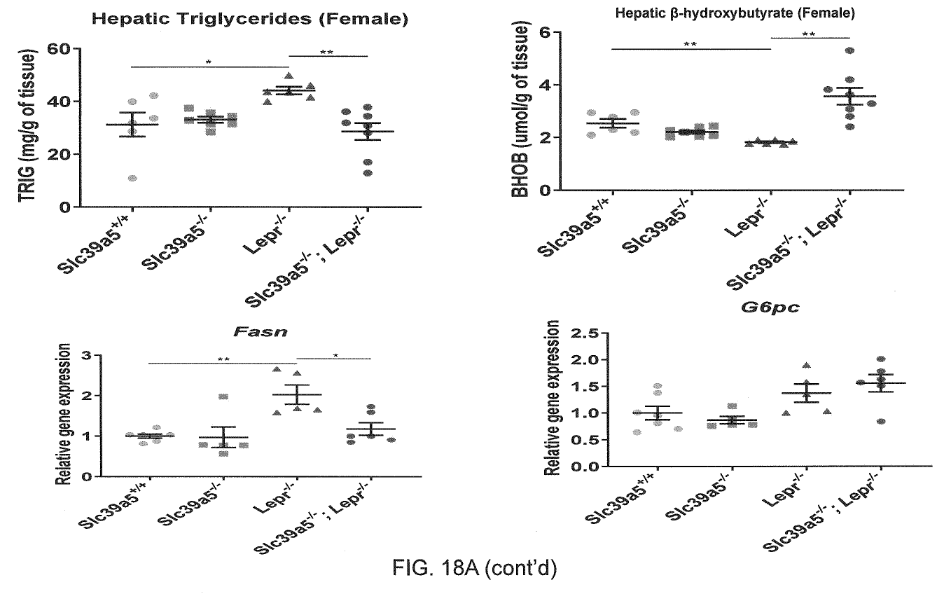

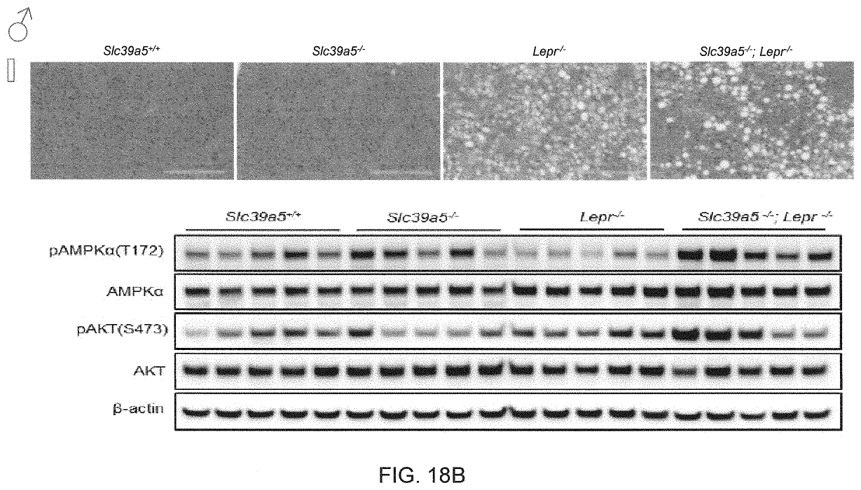

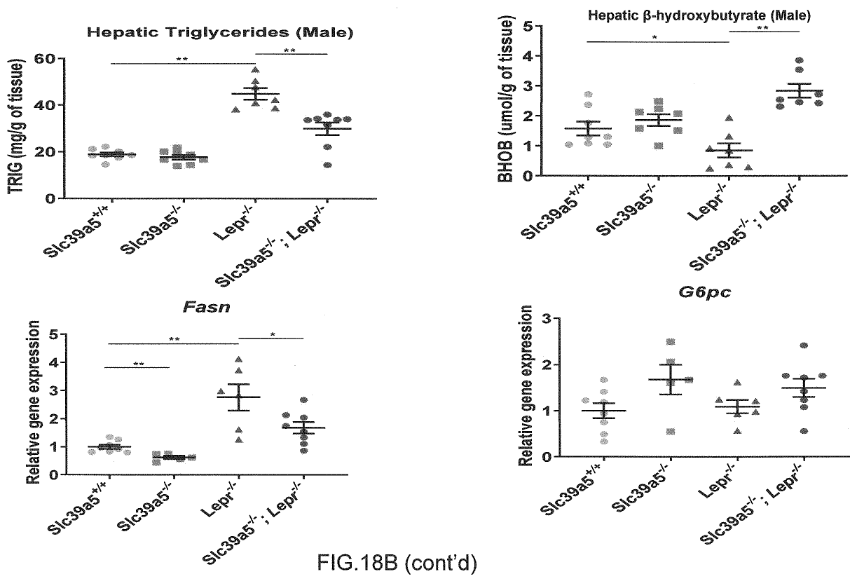

Liver protein was extracted using RIPA buffer (Cell signaling technology, Cat #9806) with Halt Protease & Phosphatase Inhibitor Cocktail (ThermoFisher Scientific, Cat #78440). Five microgram protein of each sample were separated in NuPAGE 4-12% Bis-Tris protein gel (Invitrogen, Cat # WG1403BOX), and transferred to nitrocellulose membrane using Trans-Blot® Turbo™ Transfer System (BioRad). Blotting was performed using the following Cell Signaling Technology antibodies: Phospho-AMPKa (Thr172) (Cell signaling technology, Cat #2535), AMPKa (Cell signaling technology, Cat #5831), 13-Actin (Sigma, Cat #5441), Phospho-AKT (Ser473) (Cell signaling technology, Cat #4060), AKT (Cell signaling technology, Cat #9272), Phospho-LKB1 (Ser428) (Cell signaling technology, Cat #3482), LKB1 (Cell signaling technology, Cat #3050), rabbit IgG conjugated to horseradish peroxidase (HRP) (Cell signaling technology, Cat #7074) and mouse IgG conjugated to HRP (Cell signaling technology, Cat #7076). Blots were developed using SuperSignal West Femto Substrate (ThermoFisher Scientific, Cat #34095). Signals were captured using ImageQuant LAS4000 (GE Healthcare). Results are shown in FIGS. 8-18B.

Summary of Experimental Diets Used in This Study

| | | | | Diet spec from manufacturer |
|---|---|---|---|---|
| | | | | Fat |
| Experiments | Diets | Sources | Vivarium | (kcal %) |
| Slc39a5: HFFD-Zn | High Fat High Fructose | TestDiet 5WRB-Yellow | 5 | 45.8 |
| | Diet (46 kcal % Fat, | | | |
| | 30 kcal % Fructose); | | | |
| | Zn 7 ppm | | | |
| | High Fat High Fructose | TestDiet 5WK9-Red | 5 | 45.8 |
| | Diet (46 kcal % fat, | | | |
| | 30 kcal % Fructose); | | | |
| | Zn 35 ppm | | | |
| | High Fat High Fructose | TestDiet 5WRC-Blue | 5 | 45.9 |
| | Diet (46 kcal % Fat, | | | |
| | 30 kcal % Fructose); | | | |
| | Zn 187 ppm | | | |
| | Control Diet; Zn 34 ppm | TestDiet 5WMC-Green | 5 | 10.2 |
| Slc39a5: HFFD | High Fat High Fructose | TestDiet 5WK9-Red | 5 | 45.8 |
| | Diet (46 kcal % Fat, | | | |
| | 30 kcal % Fructose) | | | |
| | Control Diet | TestDiet 58Y2-Yellow | 5 | 10.2 |
| Slc39a5: HFD | High Fat Diet | Research Diet D12492 | 9 | 60 |
| | (60 kcal % Fat) | | | |
| | Control Diet (V9 chow) | TestDiet 58Y2 | 9 | 10.2 |
| WT: HFD-Zn | High Fat (60 kcal | TestDiet 9GWQ | 9 | 61.6 |
| | % Fat); Zn 8 ppm | | | |
| | High Fat Diet | TestDiet 9GWP | 9 | 61.6 |
| | (60 kcal % Fat); | | | |
| | Zn 35 ppm | | | |
| | High Fat Diet | TestDiet 9GWR | 9 | 61.6 |
| | (60 kcal % Fat); | | | |
| | Zn 180 ppm | | | |
| | Control Diet (V9 chow) | TestDiet 58Y2 | 9 | 10.2 |
| Slc39a5/Lepr | Normal Chow (V5) | LabDiet 5053 | 5 | 13.2 |
| Vivarium water | | V9 water | 9 | |
| | | V5 water | 5 | |

| | Diet spec from manufacturer | | | Ion Quantification | | | |
|---|---|---|---|---|---|---|---|
| | Carbohydrates | Protein | | Zn | Fe | Zn | Fe |
| Experiments | (kcal %) | (kcal %) | Cholesterol | (ppm) | (ppm) | (ppm) | (ppm) |
| Slc39a5: HFFD-Zn | 36.2 | 18 | 197 ppm; 0.020% | 7 | 48 | 6.1 | 44.9 |
| | 36.2 | 18 | 197 ppm; 0.020% | 35 | 48 | 34 | 43.3 |
| | 36.1 | 18 | 197 ppm; 0.020% | 187 | 48 | 171 | 39.9 |
| | 71.8 | 18 | 18 ppm; 0.002% | 34 | 48 | 28 | 35.5 |
| Slc39a5: HFFD | 36.2 | 18 | 197 ppm; 0.020% | 35 | 48 | 32 | 52 |
| | 71.8 | 18 | 18 ppm; 0.002% | 34 | 48 | 28 | 53 |
| Slc39a5: HFD | 20 | 20 | 279 ppm; 0.03% | n/a | n/a | 46.3 | n/a |
| | 71.8 | 18 | 18 ppm; 0.002% | 34 | 48 | 29.3 | n/a |
| WT: HFD-Zn | 20.3 | 18.1 | 301 ppm; 0.03% | 8 | 64 | 8.3 | n/a |
| | 20.3 | 18.1 | 301 ppm; 0.03% | 35 | 64 | 32.1 | n/a |
| | 20.3 | 18.1 | 301 ppm; 0.03% | 180 | 64 | n/a | n/a |
| | 71.8 | 18 | 18 ppm; 0.002% | 34 | 48 | 29.3 | n/a |
| Slc39a5/Lepr | 62.1 | 24.7 | 141 ppm; 0.014% | 87 | 220 | 85 | 180 |
| Vivarium water | | | | | | <0.1 | |
| | | | | | | <0.1 | |

1. A rodent whose genome comprises a loss of function mutation in an endogenous rodent Slc39a5 gene at an endogenous rodent Slc39a5 locus.

2. The rodent of claim 1, wherein the mutation comprises a deletion, in whole or in part, of the coding sequence of the endogenous rodent Slc39a5 gene.

3. The rodent of claim 1, wherein the mutation comprises a deletion of a nucleotide sequence of the endogenous rodent Slc39a5 gene encoding one or more of the transmembrane domains of the Slc39a5 protein.

4. The rodent of claim 1, wherein the mutation comprises a deletion of a coding portion of exon 1 and a portion of exon 2.

5. The rodent of claim 1, wherein the mutation comprises a deletion of the nucleotide after the ATG start codon in exon 1 through the fifth nucleotide before the 3′ end of exon 2.

6. The rodent of claim 1, wherein the Slc39a5 locus further comprises a reporter gene.

7. The rodent of claim 6, wherein the reporter gene is operably linked to the endogenous Slc39a5 promoter at the Slc39a5 locus.

8. The rodent of claim 1, wherein the Slc39a5 locus comprises a deletion beginning from the nucleotide after the ATG start codon in exon 1 through the fifth nucleotide before the 3′ end of exon 2, and comprises a reporter gene coding sequence that is fused in-frame to the start (ATG) codon of the Slc39a5 locus.

9.-10. (canceled)

11. The rodent of claim 1, wherein the rodent is homozygous for the mutation.

12. The rodent of claim 1, wherein the rodent is heterozygous for the mutation.

13. The rodent of claim 1, wherein the rodent is a female rodent.

14. The rodent of claim 1, wherein the rodent is a male rodent.

15. The rodent of claim 1, further comprising a loss of function mutation in an endogenous rodent leptin receptor gene.

16. The rodent of claim 1, wherein the rodent is a mouse.

17. The rodent of claim 1, wherein the rodent is a rat.

18. A cell or tissue isolated from the rodent of claim 1, wherein the genome of the cell or tissue comprises the loss of function mutation in an endogenous rodent Slc39a5 gene at an endogenous rodent Slc39a5 locus.

19. (canceled)

20. (canceled)

21. A method of making a rodent, the method comprising
modifying a rodent genome such that the modified rodent genome comprises a loss of function mutation in an endogenous rodent Slc39a5 gene at an endogenous rodent Slc39a5 locus, and
obtaining a rodent comprising the modified genome.

22.-38. (canceled)

39. A method of identifying a Slc39a5 inhibitor, the method comprising
providing a rodent whose genome comprises a loss of function mutation in an endogenous rodent Slc39a5 gene at an endogenous rodent Slc39a5 locus,
providing a wild type rodent without the mutation,
administering a candidate Slc39a5 inhibiting agent to the wild type rodent;
examining the rodent with the mutation and the wild type rodent to measure the serum zinc levels and one or more metabolic and cardiovascular traits; and
comparing the measurements from the wild type rodent administered with the agent, from the wild type rodent before the administration of the agent, and from the rodent with the mutation to determine whether the candidate Slc39a5 inhibiting agent inhibits the activity of Slc39a5.

40.-46. (canceled)

47. The rodent of claim 11, wherein the rodent is a mouse.

48. The rodent of claim 12, wherein the rodent is a mouse.

49. The rodent of claim 13, wherein the rodent is a mouse.

50. The rodent of claim 14, wherein the rodent is a mouse.

51. The rodent of claim 1, wherein the rodent is a female mouse homozygous for the mutation.


SEQUENCE LISTING (6 sequences)

SEQ ID NO: 1 - DNA, 1980 bp, Homo sapiens
gcctgcccccccagctggaaccaagaaggttgtgtcccccttcctctgggtgtccttgtctcctgctatcagggcttcccctttcttggtcatcgatccctagagctctggctctttctcttcttggggaactgcttaacgtctacagcaaggcctaataggggacctgagggcacagtcctcaggatgtttcggggagaataggagccagaacctgagcccctaagctattcccctcaccaatgatggggtccccagtgagtcatctgctggccggcttctgtgtgtgggtcgtcttgggctgggtagggggctcagtccccaacctgggccctgctgagcaggagcagaaccattacctggcccagctgtttggcctgtacggcgagaatgggacgctgactgcagggggcttggcgcggcttctccacagcctggggctaggccgagttcaggggcttcgcctgggacagcatgggcctctgactggacgggctgcatccccagctgcagacaattccacacacaggccacagaaccctgagctgagtgtggatgtctgggcagggatgcctctgggtccctcagggtggggtgacctggaagagtcaaaggcccctcacctaccccgtgggccagccccctcgggcctggacctccttcacaggcttctgttgctggaccactcattggctgaccacctgaatgaggattgtctgaacggctcccagctgctggtcaattttggcttgagccccgctgctcctctgacccctcgtcagtttgctctgctgtgcccagccctgctttatcagatcgacagccgcgtctgcatcggcgctccggcccctgcacccccaggggatctactatctgccctgcttcagagtgccctggcagtcctgttgctcagcctcccttctcccctatccctgctgctgctgcggctcctgggacctcgtctactacggcccttgctgggcttcctgggggccctggcggtgggcactctttgtggggatgcactgctacatctgctaccgcatgcacaagaagggcggcacgcaggacctggcggactaccagagaaggacctgggcccggggctgtcagtgctcggaggcctcttcctgctctttgtgctggagaacatgctggggcttttgcggcaccgagggctcaggccaagatgctgcaggcgaaaacgaaggaatctcgaaacacgcaacttggatccggagaatggcagtgggatggcccttcagcccctacaggcagctccagagccaggggctcagggccagagggagaagaacagccagcacccaccagctctggcccctcctgggcaccaaggccacagtcatgggcaccagggtggcactgatatcacgtggatggtcctcctgggagatggtctacacaacctcactgatgggctggccataggtgctgccttctctgatggcttctccagcggcctcagtaccaccttagcggtcttctgccatgagctgccccacgaactgggtgactttgccatgctgctccagtcagggctgtcctttcggcggctgctgctgctgagcctcgtgtctggagccctgggattggggggtgcagtcctgggggtggggctcagcctgggccctgtccccctcactccctgggtgtttggggtcactgctggggtcttcctctatgtggcccttgtggacatgctaccagccctgcttcgtcctccggagcccctgcctacgccccatgtgctcctgcaggggctggggctgctgctggggggcggcctcatgcttgccataaccctgctggaggagcggctactgcccgtgaccactgagggctgatggggccagtggaaaggggtcgggttgcccttccttccccccaaccacaggaatggaggcgggacacagggccagtaggagcaataggattttaataaacagaacccatcccaaa

SEQ ID NO: 2 - protein, 540 aa, Homo sapiens
MMGSPVSHLLAGFCVWVVLGWVGGSVPNLGPAEQEQNHYLAQLFGLYGENGTLTAGGLARLLHSLGLGRVQGLRLGQHGPLTGRAASPAADNSTHRPQNPELSVDVWAGMPLGPSGWGDLEESKAPHLPRGPAPSGLDLLHRLLLLDHSLADHLNEDCLNGSQLLVNFGLSPAAPLTPRQFALLCPALLYQIDSRVCIGAPAPAPPGDLLSALLQSALAVLLLSLPSPLSLLLLRLLGPRLLRPLLGFLGALAVGTLCGDALLHLLPHAQEGRHAGPGGLPEKDLGPGLSVLGGLFLLFVLENMLGLLRHRGLRPRCCRRKRRNLETRNLDPENGSGMALQPLQAAPEPGAQGQREKNSQHPPALAPPGHQGHSHGHQGGTDITWMVLLGDGLHNLTDGLAIGAAFSDGFSSGLSTTLAVFCHELPHELGDFAMLLQSGLSFRRLLLLSLVSGALGLGGAVLGVGLSLGPVPLTPWVFGVTAGVFLYVALVDMLPALLRPPEPLPTPHVLLQGLGLLLGGGLMLAITLLEERLLPVTTEG

SEQ ID NO: 3 - DNA, 1944 bp, Mus musculus
aaggctgggtcccccttcctcttggtgtcttttgtgtctcctgtcctgccgtcagccctccttgtgacgtggcttccactgcatctgaaggctgagctccttccctcggatctcctaaggtccacagtgaggccttccagggacctgagggtacagtcctcaggatgtctcaaaggaaatagaagtcagaaacagagcctctaaccatagccctagtgatggggcccccagtacatcatctgctgactggcctatgtgtgggggtggccttgggctgggtaggaggctccgtcccaaacctgggcccagctgaacaggaacagaaccactacctggcccagttgtttggtctatatggagagaacgggacactgactgcagggggcctggcgcggcttctccacagcctaggactaggccgagtccaggggctccgtctgggacaccatgagcctccaactgggcgggctgcacccacaagtggagacaatttcacacacaggctgcaagagccggagctgagtgtggacatctgggcaggaatgcctctgggcccttcaggttggggtgaccaggaggaatcaaaggctcctgacctgcacgggtcagggccctcgagcctagacctcttccagaggcttctgctgttggaccattcgttggctgaccatctgaatgaggattgtctgaatggctcccagctgctggtcaattttggactgagccctgttgctcctctgacccctcgtcagttcgctctgctgtgcccagccctgctttatcagattgacagccgtgtttgcatcaaaaccccagctccagcacctccgggggatgtactgtctgccctgcttcacagtggcctggcagtcctgttcctcagcctccctgctcccctctctctgctgttgctgcggctcctgggacctcgtctgttgcggccagtgctgggcttcctgggggccctggccgtgggtactctctgtggggatgccctgctacacctgctcccacatgcgcaaggagggcggcacacaggacctagtgagcaatcagaggaggacctgggtccgggactgtcggtgcttggtggcctcttcctgctcttcatgctagagaacacactaggacttgtgcggcacagagggctcaggccaagatgctgcaggaacaaaagggatcttggagaaccaaaccctgacccagaggatggcagtgggatggtccttcgacccctacaggcagcttcagaaccagaggttcagggccagagggagaacagacagtcctcaccatctctagcccctcctgggcaccaaggccacagccatgagcaccggggtggcagtatcgcatggatggtcctcctgggagattgcctgcacaacctcaccgacgggctggcattaggtgctgctttctcagacggcttctccagtggcctcagcactaccctagcagtcttctgccacgagctgccccacgaactgggtgactttgccatgctgcttcaggaagggttatcctttcggaagctgctgctgctgagcctcgtttctggagccctgggactggggggtgcagccctgggggtggggctcagcttgggccctgtgcccctcaccccctgggtgtttgggaccacggctggggttttcctctatgtggccctagtggatatgctgccaaccctgcttcgtcctcctgagcccctgcctgtgttccacgtgcttctgcaggggctggggctgctactggggggcagcctcatgtttaccatagccctattggaggagcagctagtgcccacggttcctgacggctgatgtgggccagtggcagaggatccaggttgcccttccttccccccaccacaggaatggaggcgggacacagggccagtaggagcaataggattttaataaacagaacccatcccaaaaa

SEQ ID NO: 4 - protein, 535 aa, Mus musculus
MGPPVHHLLTGLCVGVALGWVGGSVPNLGPAEQEQNHYLAQLFGLYGENGTLTAGGLARLLHSLGLGRVQGLRLGHHEPPTGRAAPTSGDNFTHRLQEPELSVDIWAGMPLGPSGWGDQEESKAPDLHGSGPSSLDLFQRLLLLDHSLADHLNEDCLNGSQLLVNFGLSPVAPLTPRQFALLCPALLYQIDSRVCIKTPAPAPPGDVLSALLHSGLAVLFLSLPAPLSLLLLRLLGPRLLRPVLGFLGALAVGTLCGDALLHLLPHAQGGRHTGPSEQSEEDLGPGLSVLGGLFLLFMLENTLGLVRHRGLRPRCCRNKRDLGEPNPDPEDGSGMVLRPLQAASEPEVQGQRENRQSSPSLAPPGHQGHSHEHRGGSIAWMVLLGDCLHNLTDGLALGAAFSDGFSSGLSTTLAVFCHELPHELGDFAMLLQEGLSFRKLLLLSLVSGALGLGGAALGVGLSLGPVPLTPWVFGTTAGVFLYVALVDMLPTLLRPPEPLPVFHVLLQGLGLLLGGSLMFTIALLEEQLVPTVPDG

SEQ ID NO: 5 - DNA, 2828 bp, Rattus norvegicus
gtgacgtggcttccactgcatctgaagactgaactccctgactcagaaatctcttaaggtccacagtgaggccctccagggacctgaggtgccattaggagtgaggccctgcatatgtgggggtgaggggagtagggcggtggaacaggttaagaggtgggtcttggggatctgagatactagtctggtttctagaccagacctaaggagagctctggggtcaggaggctgctccagaaggcagaaggaaggggcagagtaagcctgaggctgcagaggctgcagaggctgcagatagactcaggtacaatcctcaggatgtcccaaaggaaatagaagtcagaaccagagcctctaacccatagccctagtgatggggccccccgtacatcatctgttggctggcctgtgtgtgggggtggccttgggctgggtagaaggctcagtccccaacctgggcccagccgaacaggaacagaaccactacctggcccagctgttcggtctatatggagagaatgggacattgactgctgggggcctggcccggcttctccacagcctaggactgggccgagttcaggggctccgcctgggacaccaagagcctccaactgggcgggctgcacccatgagtggcgacaatttcacacacaggcttcaggaactggagctgagtgtggatgtctgggcaggaatgcctctgagcccttcaggttggggtgaccaggaggactcaaaggcacatgacctgcatgggtcagggccctcgggcctagacctcttccacaggcttctgctgctggaccattcgttggctgaccatctgaacgaggattgtctgaatggttcccagctgctggtcaattttgggctgagtcctgttgctcctctgacccctcgtcagtttgctctgttgtgcccagccctgctttatcagattgacagccgtgtttgcatcaaaacccctgctccagcacctccaggggatgcactgtctgccctgcttcatagtggcctggcagtcctgttcctcagcctccctgctcccttctccctgctgctgctgcggctcttaggacctcgtctgttgcggcctgtgctgggcttcctgggggccctggctgtgggcactctttgtggggatgccctgctccacctgctgccacatgcacaaggaggacggcacacaggacctagtgagcaagcagaggaggatctgggtccagggctgtcagtgcttggtggcctcttcctgctcttcgtgttagagaatgcactaggacttgtgcggcacagagggctcaggccaaggtgtggcaggaacaaaagggatgtcgaagaaccaaaccctgacccagaggacggcactgggatggtccttcggcccctacaggcagcttcagaggttcagggccagagggaaaacagccagtccctaccatctccagcccctcccggataccaaggccacagtcatgagcaccggggcggtagtatcgcatggatggtcctcctgggagattgcctgcacaacctcaccgatgggctcgcactaggtgctgccttttccgacggcttctccagtggcctcagcactaccctagcagtcttctgccatgagctgccccatgaactgggtgactttgcaatgctgcttcaggaagggctgtcctttcggaagctgctgctgctgagcctcgtctctggagtcctggggctggggggtgcagccctgggggtggggctcagcttgggccctgtccccctcactccctgggtgtttgggatcacggctggggtcttcctctatgtggcccttgtggatatgctgccagccctgctccgtcctcctgagccccggcccctgccccatgtgcttctgcaggggctggggctgctgctggggggcagcctcatgtttactatagccctgttggaggaacagctactgcccgtggttcctgatggctgatgtgggccagtggcaaaggatgcaggctgcccttccttccccccaccacaggaacggaggcgggacacagggccagtaggagcaataggattttaataaacagaacccatcccaaagccatgactacgacagttgtacttgcaccaaaacagcatagaaaaccggggtgtagtgggagggctcaaagcaggtttggggaggacaagcatgggggagggcctggaggttgtggggtgcatcagtcttcagggatagcattgtgctttagcccaggtgggggggcagggcaaatgcaccaaggtccccatttttttctgctgccctcagcaccctggagatgcaggtatctgggcctattgctgcccaccagcattaaacacccctgaccccaacactagcaccacagggggatccaggatagggagaagggcaggagtgaggaaattgcttagagaaagattcaactagaatccagtgaattgtgctcagttctctttacttcctacaaccgagtacatgggtcacagggtggagggtgcaacgggacaggaacatgcccctccgtgccccccaacacgcctgcacacaggatggtggtgtctgcagcatcacaggtcatgcagggcatggggaaggggttcacacacacatagatgcccacagtggggaccagacagaacacccctgaatatacacagctgtacatggggaaccccaggtccccacccaaacctctcccctgtcttgctgttccccagcaggagaactgtattgctttgagagagccaccctggggctgctctgccaggcaccctcccctcccacccacccctattttggcacatctgcaaga

SEQ ID NO: 6 - protein, 533 aa, Rattus norvegicus
MGPPVHHLLAGLCVGVALGWVEGSVPNLGPAEQEQNHYLAQLFGLYGENGTLTAGGLARLLHSLGLGRVQGLRLGHQEPPTGRAAPMSGDNFTHRLQELELSVDVWAGMPLSPSGWGDQEDSKAHDLHGSGPSGLDLFHRLLLLDHSLADHLNEDCLNGSQLLVNFGLSPVAPLTPRQFALLCPALLYQIDSRVCIKTPAPAPPGDALSALLHSGLAVLFLSLPAPFSLLLLRLLGPRLLRPVLGFLGALAVGTLCGDALLHLLPHAQGGRHTGPSEQAEEDLGPGLSVLGGLFLLFVLENALGLVRHRGLRPRCGRNKRDVEEPNPDPEDGTGMVLRPLQAASEVQGQRENSQSLPSPAPPGYQGHSHEHRGGSIAWMVLLGDCLHNLTDGLALGAAFSDGFSSGLSTTLAVFCHELPHELGDFAMLLQEGLSFRKLLLLSLVSGVLGLGGAALGVGLSLGPVPLTPWVFGITAGVFLYVALVDMLPALLRPPEPRPLPHVLLQGLGLLLGGSLMFTIALLEEQLLPVVPDG